(19) United States
(12) Patent Application Publication
(10) Pub. No.: US 2024/0180217 A1
(43) Pub. Date: Jun. 6, 2024

(54) DATE SUGAR CONCENTRATES

(71) Applicants: Nasser Z.A. Khalifeh, Amman (JO); Munjed Munir Reda Sukhtyan, Amman (JO)

(72) Inventors: Nasser Z.A. Khalifeh, Amman (JO); Munjed Munir Reda Sukhtyan, Amman (JO)

(21) Appl. No.: 18/530,292

(22) Filed: Dec. 6, 2023

Related U.S. Application Data
(60) Provisional application No. 63/430,409, filed on Dec. 6, 2022.

(51) Int. Cl. A23L 27/30; A23G 3/42; A23L 19/00; A61K 36/889

(52) U.S. Cl. CPC A23L 27/33; A23G 3/42; A23L 19/09; A61K 36/889; A61K 2236/331; A61K 2236/39; A61K 2236/51; A61K 2236/53

(57) ABSTRACT

Date sugar concentrates and methods for using and making the same are provided.

### CROSS-REFERENCE TO RELATED APPLICATION(S)

This application claims the benefit of and/or is related to prior filed U.S. Provisional Patent Application No. 63/430,409, filed Dec. 6, 2022, which is hereby incorporated by reference herein in its entirety.

### COPYRIGHT NOTICE

At least a portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyright rights whatsoever.

### TECHNICAL FIELD

This disclosure relates to date sugar concentrates and methods for using and making the same.

### BACKGROUND OF THE DISCLOSURE

Previous methods and compositions for producing gummy form formulation include artificial sweeteners that are considered undesirable ingredient items.

### SUMMARY OF THE DISCLOSURE

This document describes date sugar concentrates and methods for using and making the same.

For example, a system is provided for making and/or using date sugar concentrates as disclosed herein.

As another example, a method is provided for making and/or using date sugar concentrates as disclosed herein.

As yet another example, a non-transitory computer-readable storage medium storing at least one program is provided, the at least one program including instructions, which, when executed by at least one processor of an electronic subsystem, cause the at least one processor to make and/or use date sugar concentrates as disclosed herein.

As yet another example, a user electronic device including a memory component, a communications component, and a processor coupled to the memory component and the communications component is provided, wherein the processor is configured to make and/or use date sugar concentrates as disclosed herein.

As yet another example, a method of generating a date product is provided that may include forming a first solution by mixing a supply of dates with a supply of water, forming a second solution by removing hard material of the dates from the first solution, forming a third solution by removing soft material of the dates from the second solution, forming a fourth solution by removing heavy material from the third solution, forming a fifth solution by removing over-sized material from the fourth solution, forming a sixth solution by removing suspended non-soluble solid from the fifth solution, forming a seventh solution by removing minerals from the sixth solution, and forming an eighth solution by removing a portion of water from the seventh solution.

As yet another example, a date syrup is provided that may include water and date sugar of at least 70° Brix in the water.

As yet another example, a date sugar concentrate is provided that may include date sugar in an amount of at least 68% by volume, and water in an amount of at least 30% by volume.

As yet another example, a semi-solid form for oral administration is provided that may include a hydrophilic long-chain polymer and a date sugar concentrate in an amount of at least 60% by volume, wherein the date sugar concentrate may include date sugar in an amount of at least 68% by volume and water in an amount of at least 30% by volume.

As yet another example, a gummy form formulation for oral administration is provided that may include date sugar concentrate in an amount of 1% by dry weight or greater, fruit juice concentrate in an amount of 1% by dry weight or greater, one or more hydrating materials, and a hydrophilic long-chain polymer.

This Summary is provided to summarize some example embodiments, so as to provide a basic understanding of some aspects of the subject matter described in this document. Accordingly, it will be appreciated that the features described in this Summary are only examples and should not be construed to narrow the scope or spirit of the subject matter described herein in any way. Unless otherwise stated, features described in the context of one example may be combined or used with features described in the context of one or more other examples. Other features, aspects, and advantages of the subject matter described herein will become apparent from the following Detailed Description, Figure(s), and Claim(s).

### BRIEF DESCRIPTION OF THE DRAWING(S)

The discussion below makes reference to the following drawing(s), in which like reference characters may refer to like parts throughout, and in which:

FIG. 1 is a flowchart of an illustrative process for preparing a date sugar concentrate, in accordance with some embodiments of the disclosure;

FIG. 2 is a schematic view of an illustrative system for preparing a date sugar concentrate, in accordance with some embodiments of the disclosure;

FIG. 3 is a more detailed schematic view of a subsystem of the system of FIG. 2, in accordance with some embodiments of the disclosure; and

FIG. 4 is a flowchart of an illustrative process for generating a date product, in accordance with some embodiments of the disclosure;

### DETAILED DESCRIPTION OF THE DISCLOSURE

Date sugar concentrates and methods for using and making the same are provided.

This disclosure may provide for the creation of a gummy or any other suitable forms (e.g., finished physical forms) and/or formulations (e.g., compositions of matter) that may be made mostly from date sugar concentrate without the addition of any artificial or processed sugar, which may be consumed as a candy or used as a carrier of an oral dose of pharmaceutical ingredients or health and wellness supplements. Delivery of date sugar concentrate in a semi-solid form using a hydrophilic long-chain polymer, such as pectin, gelatin, agar-agar, and/or the like, may be provided. Any suitable conventional ingredients (e.g., of a gummy) may be replaced with date sugar concentrate. Oral dosing of many materials with desirable properties and functions can be utilized (e.g., when provided in a chewable form), particularly by children and geriatric adults, due to the intrinsic nutritional value of such materials. Date sugar concentrate in itself has nutritional benefits, as it may contain a good amount of potassium, and the simple sugars naturally found in dates are quick to provide the body with energy. Methods and compositions for producing gummy or any other suitable forms and/or formulations including processed sugars and/or artificial sweeteners may be considered undesirable ingredient items. In particular, the intrinsic bitterness of certain active pharmaceutical ingredients (“APIs”) can present a major obstacle to the acceptance, compliance, and/or effectiveness of treatments including oral, chewable dosing. Poor palatability of certain materials can be overcome by nullifying undesirable tastes using flavor additives, chemical chelation (e.g., using ion exchange resins and β-cyclodextrins), and/or physical encapsulation, and/or by masking such tastes with increased sweetness. Gummy forms and/or formulations are particularly effective for enabling compliant dosing in children, as well as geriatric adults, as these forms provide a palatable, chewable base, can incorporate APIs, and/or can have low intrinsic taste response. Various embodiments of the disclosure utilize date sugar concentrate as a main ingredient that can provide a gummy form and/or formulation with sweetness as well as a favorable nutritional value. However, while gummy form formulation may provide the basis for effective dosing of active ingredients (e.g., to children, geriatric patients, and/or any other suitable consumers), the preparation of stable gummy forms and/or formulations including certain base ingredients (e.g., certain natural base ingredients) can be challenging. For example, the inclusion of date sugar concentrate in significant amounts can lead to stickiness, syneresis, and/or other negative textural properties. It can be beneficial to provide stable dosage forms with sufficient gummy properties incorporating natural ingredients, such as date sugar concentrate, in significant amounts and to methods for providing such dosage forms.

This disclosure provides a gummy form and/or formulation that may be made using date sugar concentrate as a main ingredient. This gummy form and/or formulation may be capable of delivering active ingredients to individuals, including those who may have difficulty swallowing conventional oral dosage forms (e.g., children and geriatric adults) and/or those who have an aversion to the taste of the active ingredients and/or those who have dosing fatigue to swallowing pills. The present disclosure provides formulations that may include significant amounts of date sugar concentrate with or without fruit juice concentrate.

In some embodiments, a gummy form formulation for oral administration is provided that may include date sugar concentrate in an amount of 80% or greater, with or without fruit juice concentrate in an amount of 10% or greater (e.g., apple juice concentrate with 2.5% malic acid), with or without fruit powder in an amount of 2% or greater (e.g., date powder or any suitable dehydrated fruit powder), with or without a calcium source, with or without one or more hydrating materials, with or without a potential of hydrogen (“pH”) adjuster, with or without flavoring and coloring agents, with or without an acid source, and/or with or without a hydrophilic long-chain polymer (e.g., pectin (e.g., 5%)). When present, such a calcium source can, in some embodiments, be a calcium salt, including, but not limited to, tricalcium phosphate. Tricalcium phosphate, where present, can be in an amount of about 0.5% to about 1% (e.g., about 0.75%). When present, such an acid source can be citric acid (e.g., 50% citric acid and 50% water, where around 2% of this solution may be added) and/or malic acid and/or sodium citrate (e.g., 0.75%). Various formulations may be used, including, but not limited to, a first formulation using gelatin as a functional ingredient (e.g., for the gummies to hold form), where a source of gelatin may be an animal (e.g., beef), a second formulation using pectin as a functional ingredient, where a source of pectin may be plant based, and where pectin may provide a similar functional effect to gelatin but may use an acid to activate and provide the form with an appropriate consistency, and a third formulation may use a combination of gelatin and pectin. One, some, or each of the formulations may be provided in various flavors. In some embodiments, the gelatin formulation may be more used in the candy industry while the pectin formulation may be more used in the pharmaceutical and wellness industries. For example, gelatin formulations (e.g., gelatin based gummies) may include about 60% date sugar concentrate, about 38% hydrated gelatin (e.g., 20% dry gelatin to 80% water), and about 2% citric acid solution (e.g., 50% water with 50% citric acid). Such a citric acid solution may not always be added, but may depend on the desired flavor (e.g., if a more citric fruit flavor is desired, this can be added as a functional ingredient that may increase shelf life and/or contribute to reaching a more authentic flavor). In some embodiments, such gelatin formulations may include about 2% date and/or other fruit powder, which may provide the form with more stability and/or add fiber to boost the nutritional value. In some embodiments, pectin formulations (e.g., pectin based gummies) may include about 82.25% date sugar concentrate, around 0-10% apple juice concentrate (e.g., 2.5% malic acid) or any other suitable juice concentrate, about 2.5% dried date powder, about 0.75% sodium citrate, and about 5% pectin. In some embodiments, gelatin and pectin combination formulations (e.g., combined gelatin and pectin based gummies) may include around 65-70% date sugar concentrate, about 20% hydrated gelatin, about 3% pectin, about 2% date powder, up to about 10% apple juice concentrate (e.g., 2.5% malic acid) or any other suitable juice concentrate, and about 2% citric acid solution (e.g., 50% citric acid in 50% water). These formulation descriptions may only reference base ingredients, while colorants and/or flavorings may be added at varying levels as appropriate and/or depending on the color and/or flavor desired (e.g., color and flavor are typically added at the rates of about 0.2% each, but this may vary depending on the desired outcome). After gummies or other forms are formed using such formulations, a layer of wax (e.g., Carnauba wax) may be added to give the gummies their shine and/or to prevent the gummies from sticking together in their packaging.

In certain embodiments, a date sugar concentrate within a disclosed gummy form formulation may be present in an amount of at least 60% or at least 50% (e.g., an amount of 50% to about 70%). In certain embodiments, when present, a fruit juice concentrate within a disclosed gummy form formulation may be present in an amount of about 5% to about 10% (e.g., an amount of about 0% to about 5% or about 5% to about 10%). In certain embodiments, when present, a fruit powder within a disclosed gummy form formulation may be present in an amount of at least 1% or at least 2% or at least 5% (e.g., an amount of 1% to about 5%). In certain embodiments, when present, a composition of a hydrophilic long-chain polymer component within a disclosed semi-solid (e.g., gummy) form formulation can vary and, in some embodiments, such a hydrophilic long-chain polymer may include, but is not limited to, pectin and/or gelatin and/or agar-agar (e.g., 7% (e.g., gelatin (e.g., dry weight))).

In addition to or as an alternative to the component(s) referenced above, a gummy form formulation of the disclosure may include one or more additional components of various types and characteristics. For example, in some embodiments, a gummy form formulation may include a calcium source and/or a dried fruit powder and/or one or more acid sources. In some embodiments, a gummy form formulation may include one or more food-grade additives, including, but not limited to, flavorants, colorants, fiber, pH-adjusters, and/or other suitable functional food ingredients. A gummy form formulation can contain any suitable active ingredient(s) (e.g., one or more vitamins, minerals, phytonutrients (e.g., carotenoids, flavonoids, resveratrol, and/or glucosinolates), fiber, fatty acids, amino acids, polypeptides, botanicals, cannabinoids, and/or the like). A gummy form formulation may include no added sucrose or corn syrup or tapioca syrup. In certain embodiments, a gummy form formulation may be sanded (e.g., coated), where such coating may include a vegan protein source on the exterior thereof.

For example, gummy form formulations of the disclosure may include, without limitation, the following embodiments:

Embodiment 1: A gummy form formulation form (e.g., for oral administration) including: date sugar concentrate in an amount of 50-85% or greater; fruit juice concentrate in an amount of 0-10% or greater; one or more hydrating materials; and a hydrophilic long-chain polymer (e.g., 5%), with an optional calcium source; and/or one or more optional food-grade additives.

Embodiment 2: The gummy form formulation of the preceding embodiment, wherein the calcium source is a calcium salt.

Embodiment 3: The gummy form formulation of any preceding embodiment, wherein the calcium source is tricalcium phosphate.

Embodiment 4: The gummy form formulation of any preceding embodiment, wherein sodium citrate is present in an amount of about 0.5% to about 1% (e.g., 0.75%).

Embodiment 5: The gummy form formulation of any preceding embodiment, wherein tricalcium phosphate is present in an amount of about 0.5% to about 0.75%.

Embodiment 6: The gummy form formulation of any preceding embodiment, wherein tricalcium phosphate is present in an amount of about 0.5% to about 1% (e.g., 0.75%).

Embodiment 7: The gummy form formulation of any preceding embodiment, wherein the date sugar concentrate is present in an amount of at least 50% or at least 60% at least 65% or at least 70%.

Embodiment 8: The gummy form formulation of any preceding embodiment, wherein the date sugar concentrate is present in an amount of at least 82.25%.

Embodiment 9: The gummy form formulation of any preceding embodiment, wherein the date sugar concentrate is present in an amount of about 50% to about 85%.

Embodiment 10: The gummy form formulation of any preceding embodiment, wherein the fruit juice concentrate is present in an amount of about 1% to about 10%.

Embodiment 11: The gummy form formulation of any preceding embodiment, wherein the fruit juice concentrate is present in an amount of about 5% to about 10%.

Embodiment 12: The gummy form formulation of any preceding embodiment, further including a fruit powder, wherein the fruit powder is present in an amount of about 1% to about 5%.

Embodiment 13: The gummy form formulation of any preceding embodiment, further including a fruit powder, wherein the fruit powder is present in an amount of about 2% to about 5% (e.g., 2.5%) by dry weight.

Embodiment 14: The gummy form formulation of any preceding embodiment, wherein the hydrophilic long-chain polymer includes pectin and/or gelatin and/or agar-agar, and/or wherein the hydrophilic long-chain polymer is present in an amount of about 5% to about 10% by dry weight.

Embodiment 15: The gummy form formulation of any preceding embodiment, wherein the gummy form formulation is free of sucrose and/or any processed sugar and/or any artificial sweetener.

Embodiment 16: The gummy form formulation of any preceding embodiment, wherein the gummy form formulation is free of corn syrup and/or tapioca syrup.

Embodiment 17: The gummy form formulation of any preceding embodiment, wherein the one or more food-grade additives is selected from the group consisting of flavorings, colorants, fiber, and pH-adjusters, and/or wherein the one or more food-grade additives is present in an amount of about 1% to about 5% by dry weight. Such additives may include, but are not limited to, natural or artificial flavoring or coloring additives, such fiber may come from fruit (e.g., fruit powder) and/or from “citrus fibers,” which may also be added as an acid source, where an acid source may be useful when using pectin. The addition of an acid may be considered a pH adjuster.

Embodiment 18: The gummy form formulation of any preceding embodiment, further including one or more vitamins.

Embodiment 19: The gummy form formulation of any preceding embodiment, further including one or more minerals.

Embodiment 20: The gummy form formulation of any preceding embodiment, further including one or more active pharmaceutical ingredients.

Embodiment 21: The gummy form formulation of any preceding embodiment, further including one or more active nutritional ingredients.

Embodiment 22: The gummy form formulation of any preceding embodiment, further including one or more active cannabinoid ingredients.

Embodiment 23: The gummy form formulation of any preceding embodiment, further including a sanded coating including protein powder from an animal or plant source.

These and other embodiments, features, aspects, and advantages of the disclosure will be apparent from a reading of the entirety of the disclosure. The disclosure includes any combination of two, three, four, or more embodiments or features or elements set forth in this disclosure or recited in any one or more of the claims, regardless of whether such embodiments or features or elements are expressly combined or otherwise recited in a specific embodiment description or claim herein. The disclosure is intended to be read holistically such that any separable features or elements of the disclosure, in any of its aspects and embodiments, should be viewed as intended to be combinable, unless the context of the disclosure clearly dictates otherwise.

The disclosure relates to gummy forms for oral use that can be suitable for the delivery of active and/or nutritional ingredients in a manner that may be highly palatable and that can provide compliance with dosing requirements for any suitable active ingredients. The disclosure, in particular, relates to gummy forms that may include a significant amount of natural ingredients (e.g., ingredients that may be obtained from natural matter (e.g., plant matter) and that are not derived from synthetic processes). In particular, certain gummy forms disclosed herein may contain a significant amount of date sugar concentrate with or without fruit juice concentrate and may exhibit good physical stability. The disclosure also relates to methods of preparing such gummy forms and to methods of using such gummy forms.

A “gummy” or “gummy form” or “gummy formulation” as used herein may refer to a confectionary that can be defined by its compositional nature, as otherwise described herein, and/or by its chewy texture and mouthfeel. Gummy bears, gummy worms, and other gummy candies are particular types of gummy, and a person of ordinary skill in the art would understand the term “gummy” may refer to a composition having such texture and mouthfeel. It is noted that the gummy forms disclosed herein may vary somewhat in texture and mouthfeel. All such textures and mouthfeels are intended to be included within the general definition of “gummy.”

The “active ingredient” included within the gummy forms disclosed herein can be any compound, composition, or like material that may be included in a dosage form for delivery to an individual to achieve any one or more of a desired nutritional purpose, medicinal purpose, and therapeutic purpose. The types of active ingredients incorporated within the disclosed gummy forms may include, but are not limited to, vitamins, minerals, phytonutrients (e.g., carotenoids, flavonoids, resveratrol, and glucosinolates), fiber, fatty acids, amino acids, polypeptides, botanicals, and/or cannabinoids. Further, non-limiting examples of materials that may be included as an active ingredient include APIs, and non-limiting examples of APIs may include nonsteroidal anti-inflammatory drugs (“NSAIDs”) (e.g., ibuprofen, diclofenac, and naproxen), analgesics (e.g., acetaminophen, aspirin), antihistamines, decongestants, antitussives, expectorants, sleep aids, antibiotics, laxatives, anti-diarrheals, anthelmintics, and antacids. An active ingredient can include any plant-derived material that is safe for human consumption, including, but not limited to, herbal extracts, botanical extracts, and the like. Other materials, such as prebiotics and probiotics, can also be used as an active ingredient. In some embodiments, an active agent according to the present disclosure may be classified as dietary supplement according to the Dietary Supplement Health and Education Act of 1994, whereby a dietary supplement may be defined to mean a product intended to supplement the diet that bears or contains one or more of the following dietary ingredients: a vitamin, a mineral, an herb or other botanical, an amino acid, a dietary substance for use by a person to supplement their diet by increasing the total dietary intake, or a concentrate, metabolite, constituent, extract, or combination of any of the aforementioned ingredients. In certain embodiments, the active ingredient may include a combination of such components (e.g., in the form of a multivitamin, including more than one vitamin). The relative amounts of the vitamins can vary and may be such that the resulting gummy form formulation is suited generally for adults (including specific formulations for men and women) or children. The specific active ingredients and amounts of a given dosage form can be designed for specific benefits (e.g., to promote immune health or to provide prenatal benefits). Certain active ingredients for inclusion within a multivitamin gummy form and/or formulation as disclosed herein may include any one or any combination of two or more of Vitamin A, Vitamin C, Vitamin D3, Vitamin E, thiamin, riboflavin, niacin, Vitamin B6, folic acid, Vitamin B 12, biotin, pantothenic acid, calcium, iodine, zinc, choline, and inositol (e.g., wellness gummies may be a carrier for vitamins, minerals, and/or nutritional supplements of any appropriate type(s)).

The gummy forms disclosed herein may further include, in addition to the one or more active ingredients, one or more components, including, but not limited to, gellants, hydrating source(s), colorants, and/or flavorants. Gummy forms may include one or more hydrocolloid systems, which can include, in some embodiments, one or more hydrophilic long-chain polymers, one or more hydrophilic bulking agents, and/or a hydrating material (e.g., water and/or a fruit concentrate). Optionally, such a hydrocolloid system can include one or more further ingredients, such as pH modifiers, coloring agents, and/or flavoring agents. A gummy form formulation can further include one or more ions or ion sources, which can, in some embodiments, function to “set” the gummy form.

Hydrophilic, long-chain polymers useful in such hydrocolloid systems may include, but are not limited to, long chain carbohydrates (e.g., polysaccharides) as well as various proteins. Such a hydrophilic, long-chain polymer preferably may be configured to thicken and form a gel upon hydration (with or without heating). Non-limiting examples of hydrophilic, long-chain polymers that may be included in a hydrocolloid system for use within the gummy forms disclosed herein include gelatin, pectin (e.g., including modified and unmodified forms of pectin), carrageenan, gellan gum, locust bean gum, gum arabic, xanthan gum, starch, methylcellulose, agar, konjac, alginates, and/or combinations thereof (e.g., including single, binary, tertiary, or quaternary blends).

In certain disclosed gummy forms, at least a portion of a hydrocolloid system of the dosage forms may include pectin. Pectin is a heteropolysaccharide that can be rich in galacturonic acid. Pectin is available in both high methoxyl and low methoxyl forms, wherein the reference to “methoxyl” refers to the number of carboxyl groups on the pectin backbone that are methyl esters. “Low-methoxyl” pectin may be understood to include less than about 50% esterified carboxyl groups, and “high-methoxyl” pectin, by contrast, may be understood to include greater than about 50% esterified carboxyl groups. In certain embodiments herein, pectin may be incorporated in high-methoxyl pectin form. The amount of pectin within the disclosed gummy forms may be less than about 10% by dry weight (e.g., in certain embodiments, less than about 7% by dry weight, less than about 5% by dry weight, less than about 4% by dry weight). An exemplary range of pectin content with respect to certain embodiments is about 3% to about 5% pectin by dry weight or about 0% to about 3% pectin by dry weight. In certain disclosed gummy forms, at least a portion of a hydrocolloid system of the dosage forms may include gelatin. Typically, gelatin may be included in an amount of at least about 10% by dry weight or at least about 7% by dry weight (e.g., about 5% to about 7% by dry weight or about 0% to about 10% by dry weight).

In certain embodiments, a hydrophilic long-chain polymer component in the disclosed gummy forms may include a combination of gelatin and pectin. The ratio of gelatin to pectin can vary, but in certain preferred embodiments, the overall amount of gelatin by dry weight in the disclosed gummy forms may be greater than the overall amount of pectin by dry weight. Certain exemplary weight ratios of these two components may include about 1:1 to about 20:1 (e.g., about 5:1 to about 20:1 or about 10:1 to about 20:1). Advantageous relative amounts of these components may be affected, in some embodiments, by any active ingredient(s) contained within the gummy form formulation.

In some embodiments, at least a portion of a hydrophilic bulking agent in the disclosed compositions may include a date derivative. A date derivative, as used herein, is a natural substance produced (e.g., by extracting date sugars) from date fruit. Date sugar concentrate generally may contain glucose and fructose, as well as trace enzymes, vitamins, minerals, and/or amino acids. Date sugar concentrate, as may be produced by a process of concentration through evaporation, may be a common and commercially available date derivative and may be advantageously incorporated within gummy forms as disclosed herein.

Date sugar concentrate may be available in various forms and concentrations based on color (e.g., ranging from “water white” to “dark amber”) and quality (e.g., Grade A, Grade B, Grade C, or “Substandard”), and any such date sugar concentrate can be used within the disclosed gummy forms. Another distinguishing characteristic of date derivative that can be used to classify different grades may be its appearance/form, which can be correlated with processing treatments to which it has been subjected. For example, date syrup may be minimally processed and may not be subjected to excessive filtration. Crystallized date syrup may include at least some crystalized date sugars, which may, for example, be returned to liquid form by heating (e.g., with mild agitation). Filtered date syrup may be date syrup that has been subjected to filtration processes to remove suspended date micro-fibers and much of the minerals naturally found in dates. This can result in largely removing the pigmentation (e.g., some or most of the natural date pigmentation) and/or flavor (e.g., some or most of the natural date flavor) when providing the resulting syrup (e.g., date sugar concentrate) Filtered date syrup, which may also be referred to as liquid sugar or decolorized date syrup or golden date syrup or pure date juice, may be filtered, optionally using enzymes, to remove more materials, including fine particles, date pigmentation and aroma, fine date fibers, and/or the like, and/or may be further filtered (e.g., using an absorbent resin or activated carbon). This type of date syrup may to a high degree not demonstrate the typical form or function of date syrup, where the natural date flavor and aroma may be removed to a great degree, thereby leaving the natural date sugars as well as some of the vitamins and minerals left in the solution.

Although any of the date derivative classes described herein can be included within the disclosed gummy form formulation, in certain embodiments, the gummy forms disclosed herein and, in particular, at least a portion of a hydrophilic bulking agent may beneficially include a date derivative.

Advantageously, a hydrophilic bulking agent in certain disclosed gummy compositions may include at least about 30% date sugar concentrate, at least about 40% date sugar concentrate, at least about 50% date sugar concentrate, at least about 60% date sugar concentrate, at least about 70% date sugar concentrate, or at least about 80% date sugar concentrate, based on the dry weight of hydrophilic bulking agents present in the gummy form and/or formulation.

Further, certain gummy forms provided herein may include one or more fruit or vegetable juice concentrates. Fruit and vegetable juice concentrates (herein referred to simply as fruit juice concentrates) may include juice from any one or more fruits and/or vegetables, which may have been processed so as to remove at least a portion of the moisture therefrom. Such fruit juice concentrates may be typically concentrated to a Degrees Brix of about 10 or greater, or 20 or greater, or 30 or greater, or 40 or greater, or 50 or greater, or about 60 or greater. In some embodiments, the date sugar concentrate may be concentrated to above 72 Brix, which may result in a syrup consistency. At 72 Brix, the product may be most stable and provide for a long shelf life with minimal risk of fermentation due to contamination. Brix is a unit of measurement of sugar content in an aqueous solution and 1 percent or 1 degree Brix (Bx) is defined as 1 gram of sucrose in 100 grams of solution. Brix measurements are generally made by measuring the specific gravity of the solution/slurry using various instruments including, but not limited to, hydrometer, refractometer, pycnometer, or U-tube meter. The specific gravity can be converted to Bx, for example, using the Brix Table maintained by the National Institute of Standards and Technology.

Fruit juice concentrates may include, but are not limited to, juices from apple, apricot, banana, blackberry, black currant, black raspberry, blueberry, boysenberry, grape, grapefruit, cranberry, cherry, elderberry, kiwi, guava, mango, passion fruit, peach, nectarine, pear, plum, pomegranate, red currant, red raspberry, blue raspberry, strawberry, watermelon, lime, lemon, orange, pineapple, and any combinations thereof. Vegetable juice concentrates may include, but are not limited to, juices from butternut squash, tomato, celery, cucumber, kale, carrot, pumpkin, beet, rhubarb, ginger, celery, and any combinations thereof. If present, the amount of juice concentrate incorporated within certain disclosed gummy forms can vary, but may typically be at least about 10%, such as about 5% to about 10% (e.g., about 1% to about 5% or about 5% to about 10%).

As such, gummy forms disclosed herein advantageously may include a combination of date sugar concentrate and one or more juice concentrates, and/or one or more fruit powders, and/or, in particular embodiments, decolorized date syrup, and/or fruit powder. In some embodiments, a gummy form formulation may include no “added sugars” other than the natural sugars found in the date syrup and/or fruit powder and/or fruit juice concentrate. “Added sugars” as used herein may refer to sugars other than sugars from natural sources (e.g., the sugars in the date syrup and the sugars in the juice concentrate and the sugars in the fruit powder), and includes such sugars added in solid or liquid (e.g., syrup) form. A hydrating material (e.g., water source) of a hydrocolloid system can include any variety of materials configured to donate water to a hydrophilic, long-chain polymer. A hydrating material particularly can be substantially pure water. However, the hydrating material may be an aqueous composition that may include one or more additives, such as a syrup, a fruit juice, or a flavoring liquid.

Gummy forms of the disclosure may include a calcium source (e.g., tricalcium phosphate (“TCP”) or calcium carbonate). Other components that serve a similar function within the gummy forms can, in some embodiments, be used in place of or in combination with the calcium source. In some embodiments, tricalcium phosphate may be employed, and may be present within a gummy form in an amount of at least about 0.75% (e.g., about 0.5% to about 1%). In some embodiments, this component may help in decreasing the “stickiness” associated with certain gummy form formulation compositions.

In some embodiments, a pH modifier may be included in a hydrocolloid system of a gummy, which, particularly can be a buffer or acidifier. Non-limiting examples of buffer and/or acidic materials that can be used include citric acid, sodium citrate, malic acid, lactic acid, tartaric acid, fumaric acid, phosphoric acid, ascorbic acid, sodium bisulfate, and/or combinations thereof. Flavoring agents can by natural or artificial (e.g., but not artificial sweeteners) and can include, but are not limited to, citric acid, tartaric acid, salts (e.g., sodium chloride), plant extracts (e.g., vanilla, luo han guo, etc.), vegetable juice (e.g., carrot concentrate), pulp, and/or extracts, fruit juice concentrate, pulp, zest, and/or extracts (e.g., apple, strawberry, raspberry, blackberry, blueberry, etc.), nuts, seeds, warm sensation materials (e.g., fats or fat rich ingredients, such as cocoa, cocoa butter or mass, vegetable oils, nut butter, etc.), cool sensation materials (e.g., menthol, cooling additives, etc.), tingling sensation materials (e.g., sour salts, etc.), and/or essential oils.

In a non-limiting example (e.g., for a vegan (e.g., pectin) gummy), a gummy form formulation may include about 50% to about 85% of decolorized date syrup, about 5% to about 10% fruit juice concentrate, about 1% to about 5% fruit powder, about 0% to about 0.75% tricalcium phosphate, about 0.5% to about 0.75% sodium citrate, about 1% to about 2% citric acid solution, and about 3% to about 5% pectin, based on the total dry weight of the gummy form and/or formulation. In another non-limiting example (e.g., for a gelatin gummy), a gummy form formulation may include about 50% to about 70% of decolorized date syrup, about 5% to about 10% fruit juice concentrate, about 1% to about 5% fruit powder, about 1% to about 2% citric acid solution, and about 5% to about 10% gelatin, based on the total dry weight of the gummy form and/or formulation. The gummy forms provided according to the present disclosure may, thus, generally include a significant amount of date sugar concentrate in addition to one or more active ingredients, as described herein.

In certain embodiments, a gummy form formulation may be provided with active ingredient(s) that may be substantially homogenously distributed throughout the dosage form. In particular, an active ingredient may be substantially homogeneously distributed throughout a hydrocolloid system of the gummy. The gummy forms may include an outer coating or may be uncoated. The gummy forms can be provided in various sizes, shapes, geometries, and/or total weight. Exemplary gummy forms as disclosed herein can be provided with masses including, but not limited to, masses in the range of about 1 gram to about 6 grams, or about 2 grams to about 5 grams. Such “forms” may be provided in various sizes depending on the intended application. For example, candy gummies may be provided as 1.5-2.0 grams each, while nutritional or wellness or supplement carrying gummies may be larger (e.g., around 6 grams each) (e.g., in order to conceal the off-taste of the active ingredients in more sweetness). The gummy forms can be generally homogeneous with respect to all components thereof or can include one or more components in a non-homogeneous association with remaining components. For example, a gummy form formulation may be provided wherein one or more components may be only partially blended into the composition (e.g., so as to produce the effect of a visual “swirl” of colorant and/or flavorant on and/or within the composition). Such “swirls” and other patterned non-homogeneities of colorants, flavorants, and/or other components are intended to be encompassed by the present disclosure.

The gummy forms provided herein generally can be characterized as being elastic or viscoelastic materials, and can be described as substantially chewable. A “chewable” dosage form, while capable of being swallowed whole, is configured specifically for chewing prior to swallowing. As such, a chewable dosage form may be specifically distinguishable from a non-chewable dosage form, such as a vitamin tablet or capsule that is intended to be swallowed whole. In some embodiments, the term chewable can thus mean that the dosage form is intended to be retained in the mouth of the consumer for a period of time prior to swallowing, during which time the dosage form may undergo a change in structure that facilitates ease of swallowing. The chewable dosage form may thus be reduced to smaller pieces through mastication. In some embodiments, the chewable dosage form may be configured to dissolve at least partially within the mouth of the consumer. As such, the chewable dosage form may also be dissolvable and may thus be referred to as a “melt-away” form.

It is understood that the oral dosage forms of the present disclosure may be configured for undergoing changes under various mouth conditions. Discussion herein of “mouth conditions” can relate to one or more characteristics (in any combination) associated with the presence of an item in the mouth of an individual. For example, mouth conditions can include any combination of temperature, moisture, and pH typically found in the mouth of a consumer as well as the shear, compression, and other mechanical forces that may be applied by the teeth during chewing. Mouth conditions can particularly relate to being in contact with saliva. In some embodiments, mouth conditions can particularly mean contact with saliva at the temperature and pH typically present in the mouth of a consumer (e.g., a human).

Advantageously, the disclosed dosage forms may include an optional sanded coating, and preferably on all exposed surfaces of the gummy form formulation. Sanded coatings may include particulate materials (e.g., including particulate/powdered fruit or vegetables, particulate plant based protein, or combinations thereof) that, in the context of the disclosed gummy form formulation, may be coated on and adhere to the surface thereof by electrostatic forces and/or mechanical adherent properties. The composition of the sanded coating can vary and, in certain embodiments, may be a protein (e.g., about 2% by weight or more protein, about 5% by weight or more protein, about 10% by weight or more protein (e.g., about 2-10% by weight protein)). The remainder of the sanded coating can include powdered fruit or vegetables as disclosed herein, which may, in some embodiments, modify the specific taste characteristics (e.g., the inclusion of certain acids can provide a sour flavor on the exterior of the gummy form formulation). Overall, the sanded coating, where present, may include a relatively small percentage of the sanded gummy form formulation (e.g., about 1% or less by weight of the sanded gummy form formulation or about 2% or less by weight of the sanded gummy form formulation).

The disclosed gummy forms advantageously may exhibit high physical stability. Stability in this sense is understood to refer to minimal stickiness and/or minimal syneresis. Such physical characteristics can be evaluated in various ways (e.g., by physical evaluation, which preferably indicates little to no change in the shape of the gummy form formulation over a period of days (e.g., for about 10 days or more, about 30 days or more, about 60 days or more, about 6 months or more, after a year or more, or after 2 years or more). Any combination of components (e.g., date sugar concentrate, fruit juice concentrate, gelatin, pectin, calcium source, fruit and/or vegetable powder and plant based protein) and amounts thereof as identified herein may uniquely provide a highly stable dosage form wherein decolorized date syrup may be the principal component. Advantageously, the disclosed dosage forms may exhibit extended flavor release and, texturally, minimal tooth-stick.

In preferred embodiments, a plurality of the disclosed gummy forms may “flow freely,” whereby they do not stick to one another to any significant extent. As such, in preferred embodiments, the disclosed gummy forms may not be difficult to extract from a container due to interaction/sticking between multiple such dosage forms.

Certain exemplary gummy forms as disclosed herein may include the components in amounts as follows in Tables 1, 2, or 3:

TABLE 1
Exemplary Gummy Formulation

| Decolorized | Fruit Juice | | | | | Sanded | Citric | Sodium | Malic |
|---|---|---|---|---|---|---|---|---|---|
| Date Syrup | Concentrate | Water | Gelatin | Pectin | Calcium | Coating | Acid | Citrate | Acid |
| 15-85% | 15-85% | 0-32% | 0-15% | 0-15% | 0-15% | 0-10% | 0-5% | 0-5% | 0-15% |

*At least some gelatin and/or pectin included to provide a gummy form formulation.

TABLE 2
Another Exemplary Gummy Formulation (Pectin-Based)

| Decolorized | Fruit Juice | | Fruit | | Sanded | Citric | Flavoring | Coloring | Acid |
|---|---|---|---|---|---|---|---|---|---|
| Date Syrup | Concentrate | Water | Powder | Pectin | Coating | Acid | Additives | Additives | Source |
| 15-85% | 15-85% | 0-32% | 0-5% | 3-10% | 0-10% | 0-5% | 0-3% | 0-3% | 0-15% |

TABLE 3
Another Exemplary Gummy Formulation (Gelatin-Based)

| Decolorized | Fruit Juice | | Fruit | | Sanded | Citric | Flavoring | Coloring |
|---|---|---|---|---|---|---|---|---|
| Date Syrup | Concentrate | Water | Powder | Gelatin | Coating | Acid | Additives | Additives |
| 15-85% | 15-85% | 0-32% | 0-5% | 5-10% | 0-10% | 0-5% | 0-3% | 0-3% |

The term “dry weight” as used herein may be based on and/or mean the total weight of the finished product (i.e., the gummy form). As such, all reported amounts and percentages are to be construed accordingly. The values stated in Tables 1-3 may cover the entire spectrum between dry weight and weight by volume (e.g., some ingredients may be considered wet (e.g., moisture may contribute to the overall composition).

The disclosure also provides methods for preparing gummy forms. Specifically, the disclosed methods may involve operations of preparing a hydrocolloid system slurry, optionally including one or more active ingredients, heating the hydrocolloid system slurry to thicken the slurry (e.g., and specifically to achieve a particular Brix level) and subsequently adding the one or more additional ingredients thereto to provide a gummy form formulation.

Generally, a slurry including a date sugar concentrate and a hydrocolloid system (e.g., including a hydrophilic long-chain polymer, hydrophilic bulking agent, hydrating ingredient, and/or other optional component(s) as disclosed herein) may be prepared. The components can be combined in various orders. For example, in one embodiment, at least a portion of a hydrophilic long-chain polymer may be added to water including sodium citrate (e.g., at elevated temperature) to give a first mixture. In one particular embodiment, sodium citrate and a foam suppressor may be combined and heated up to 250ºF, and pectin, water, and gelatin may be added thereto, thereby giving a first mixture. This first mixture can be stirred and, optionally, heated to allow for dissolution of the hydrophilic long-chain polymer. To this first mixture, certain remaining components, including, but not limited to, fruit juice concentrate and malic acid, citric acid and flavorants, colorants and other functional ingredients, and/or the like, may then be added. Advantageously, such components can be pre-combined and heated at elevated temperature (e.g., up to 250° F.) and this second mixture may be added to the first mixture. Subsequently, all remaining components and certain active ingredients may then be added. The resulting slurry including the hydrocolloid system may be heated/cooked to the desired concentration. The temperature can vary, and, in one particular embodiment, may be up to 250ºF). Generally, a desired concentration can be defined by the Brix level of the slurry, which can be monitored and measured as described herein. In some embodiments, the hydrocolloid slurry, just prior to addition of the active ingredient(s), may have a Brix of at least about 50%, at least about 60%, at least about 70% (e.g., about 60-80% Brix or about 70-80% Brix (e.g., 74% Brix)), or the like.

The active ingredient(s) to be incorporated within a gummy form formulation can be added to a slurry prior to cooking (e.g., when the slurry is in a less concentrated form), so as to ensure homogeneity of the resulting product, or can be added after cooking a slurry to form a thickened slurry (e.g., when a slurry has achieved the Brix levels referenced herein). In some embodiments, solid active ingredients may be added prior to cooking, and liquid active ingredients may be added after cooking. Colors, flavors, and/or acids may also be added as a post-dose after cooking.

An active ingredient-containing, thickened slurry may be deposited into molds and cooled therein to set the final, desired shape when released from the molds to provide gummy forms. The time required to achieve the gummy forms after depositing into the molds can vary. For example, sufficient setting of a slurry may be achieved in the molds within an hour at ambient conditions (e.g., room temperature and ambient pressure) and sufficient drying of the slurry is achieved in the molds within about 72 hours, after which time the gummy forms can be removed from the molds. It is noted that certain forms (e.g., those deposited on a silicone mold) may require little to no drying time (e.g., less than about 1 hour). Advantageously, the gummy forms may exhibit sufficient integrity to remain in a desired form (e.g., without flowing) after removal from the molds. In some embodiments, the dosage forms removed from the molds can be processed (e.g., by applying oil or anti-sticking agents thereto and/or by applying a coating). In particular, a sanded coating may be advantageously applied to disclosed gummy forms following removal from the molds. A solvent may be a substance (e.g., usually a liquid) that may dissolve a solute, resulting in a solution. A concentrate may be a form of substance that has had the majority of its base component (e.g., in the case of a liquid: the solvent) removed. This may be the removal of water from a solution or suspension, such as the removal of water from fruit juice. One benefit of producing a concentrate is that of a reduction in weight and volume for transportation, as the concentrate can be reconstituted at the time of usage by the addition of the solvent.

Embodiments of the present disclosure may be further illustrated by the following examples, which are set forth to illustrate the presently disclosed subject matter and are not to be construed as limiting.

As mentioned, the present disclosure relates to date sugar concentrates and/or orally ingestible gummy form formulations with date sugar concentrates and methods for making such formulations and methods for using such formulations. The gummies can contain significant amounts of date sugar concentrates with or without fruit juice concentrate.

Dates (i.e., Phoenix dactylifera) are a seasonal fruit predominantly grown in the Middle East and Gulf, as the plant is well adapted to arid climates. Date fruit, in its many known varieties, has been a cultural staple in the region. Thought to carry significant nutritional benefits as well as a very low glycemic index. Some of the proven benefits are significant vitamin and mineral content, fiber content, naturally occurring simple sugars as well as a good source for antioxidants.

Most harvested dates are good enough for direct consumption. However, lower grade dates (e.g., based on size, shape, and/or overall aesthetic) are not considered desirable for direct human consumption so other uses have been developed. If the dates are severely damaged and their shelf stability is compromised, they may be directed into the animal feed industry. All other lower grade dates may go towards making either date syrup or date paste. These two main product categories may determine the end use of the resulting product, where date paste may be used in nutrition bars, sauces (e.g., A1 steak sauce type products), baking, ice cream, and many more, while date syrup may be used as a topping or any other industrial application where the flavor and/or aroma of dates is desired. This is because of the potency of the syrup, it is concentrated to about 75% date sugars, after removing the fibers and pits.

A facility can be designed to receive lower grade dates from regional sources and make what may be called pure date juice, decolorized date syrup, date liquid sugar, golden date syrup, and/or the like. Effectively, the result may be a date sugar concentrate where the majority of the suspended fibers, ash content, pigments, and/or aroma of dates may be removed through any suitable filtration process.

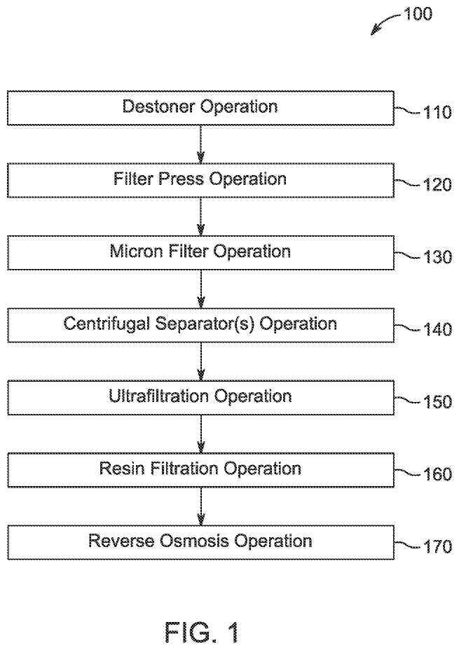

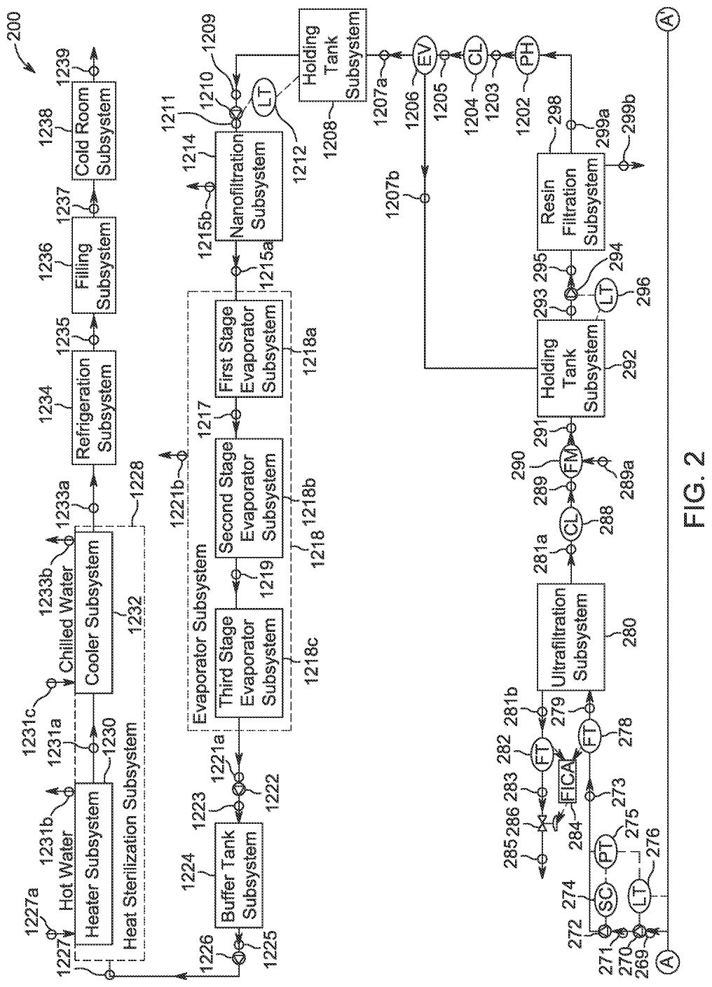

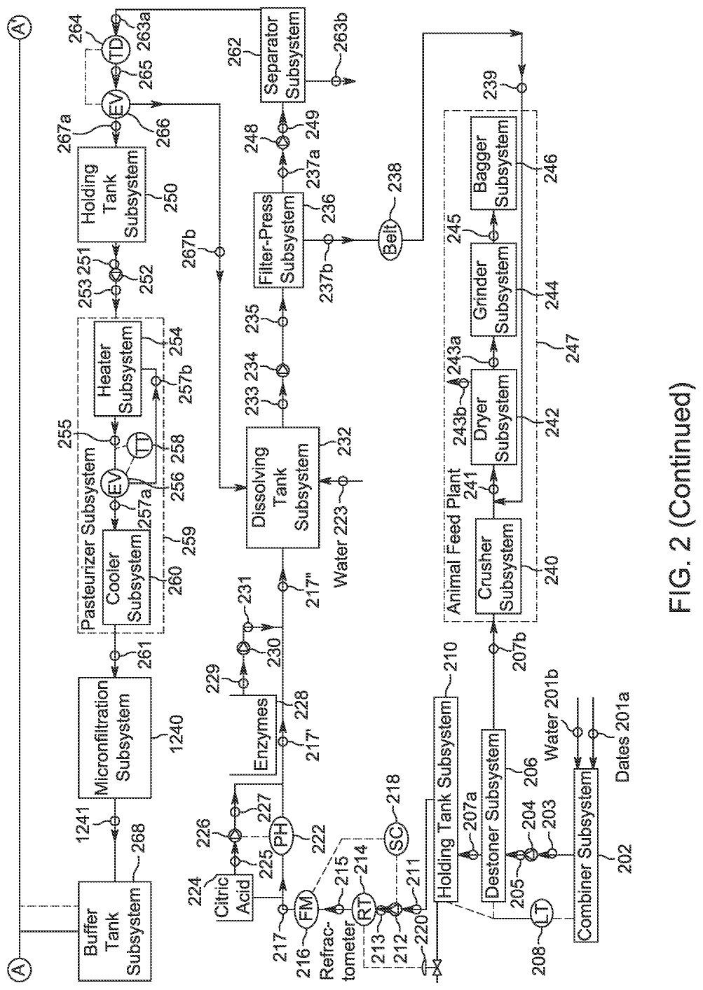

For example, as shown in FIG. 1, an exemplary process 100 may be provided for preparing date sugar concentrate from dates (e.g., for use in any suitable forms and/or formulations (e.g., gummies, juices, marshmallows, etc.) of the disclosure), where process 100 may be carried out using any suitable system(s) (e.g., system 200 of FIG. 2). At operation 110 of process 100, any suitable destoner operation(s) (e.g., any suitable mechanical process(es)) may be utilized to remove the pits (e.g., seeds) and any other suitable elements (e.g., date flesh, water, etc.) from one or more dates that may be combined (e.g., as a solution) with any suitable other material(s), such as water, in any suitable container. An enzyme may be used to breakdown the cellulose, which can allow the sugar from the dates to diffuse into the water. An enzyme may be used to breakdown the cellulose, which can allow the sugar from the dates to diffuse into the water. The pits and/or any other suitable hard material may be removed (e.g., mechanically) through the destoner (e.g., a perforated stainless steel drum). The liquid, pits, and fiber may pass through the destoner (e.g., drum), where the liquid and fiber may be pushed through the perforations while the pits and any other hard material may be guided through a different exit to be collected.

At operation 120 of process 100, any suitable filter press operation(s) (e.g., any suitable mechanical process(es)) may be utilized to remove any suitable date flesh and/or soft material of the solution (e.g., date-sugar saturated water) that may survive operation 110 (e.g., that may go through the destoner (e.g., calyxes)). This operation may be configured to remove the fibers from the date-sugar saturated water.

At operation 130 of process 100, any suitable micron filter operation(s) (e.g., any suitable mechanical process(es)) may be utilized (e.g., as a pass through filter) to catch any over-sized material that may survive operation 120 (e.g., that may come through a tear/malfunction in the filter press of operation 120). This operation may be configured to remove the microfibers (e.g., very small pieces of date fiber) that may be suspended in the solution (e.g., date-sugar saturated water) that may survive operation 120.

At operation 140 of process 100, any suitable centrifugal separator operation(s) (e.g., any suitable mechanical process(es)) may be utilized to spin the solution at very high speeds and rely on centrifugal force to pull the heavier materials (e.g., contaminants) of the solution to an outer rim where they may be captured and discarded while the lighter (e.g., clean) juice of the solution may be retained (e.g., by flowing through a center of the separator). In some embodiments (e.g., as may be shown with respect to system 200 of FIG. 2), operation 140 may occur after operation 120, and operation 130 may occur after operation 140, where, at operation 140 of process 100, any suitable centrifugal separator operation(s) (e.g., any suitable mechanical process(es)) may be utilized to spin the solution (e.g., solution that may survive operation 120 (e.g., that may come through a tear/malfunction in the filter press of operation 120)) at very high speeds and rely on centrifugal force to pull the heavier materials (e.g., contaminants) of the solution to an outer rim where they may be captured and discarded while the lighter (e.g., clean) juice of the solution may be retained (e.g., by flowing through a center of the separator), and, then, at operation 130 of process 100, any suitable micron filter operation(s) (e.g., any suitable mechanical process(es)) may be utilized (e.g., as a pass through filter) to catch any over-sized material that may survive operation 140, where operation 130 may be configured to remove the microfibers (e.g., very small pieces of date fiber) that may be suspended in the solution (e.g., date-sugar saturated water) that may survive operation 140.

At operation 150 of process 100, any suitable ultrafiltration operation(s) (e.g., any suitable mechanical process(es)) may be utilized (e.g., with a network of membrane fitted tubing) to remove micron-sized particles from the solution. Such ultrafiltration may be configured to remove any suspended non-soluble solids that are too small to be filtered out by the filter press (e.g., of operation(s) 120). For example, such ultrafiltration operation(s) 150 may be one or more subprocesses wherein sub-micron particles may be filtered out. During (e.g., at the end of) operation(s) 150, even some of the mineral content naturally found in dates may be removed. While one or some or all of operation(s) 150 may be useful in various manufacturing operations, such as dairy and beverage manufacturing, these operation(s) 150 may be applied to the processing of dates for the purpose of decolorizing and/or de-odorizing the resulting syrup.

At operation 160 of process 100, any suitable resin filtration operation(s) (e.g., any suitable mechanical process(es)) may be utilized (e.g., with adsorbent resin (e.g., Dowex Optipore SD-2 Adsorbent by the Dow Chemical Company and/or AmberLite SD-2 Polymeric Adsorbent by DuPont de Nemours, Inc.)) to decolorize and vastly reduce the turbidity of the solution. Resin filtration operation(s) 160 may be configured to remove the minerals that give date juice its date taste and aroma, such that operation(s) 160 may result largely in the removal of the natural color and/or taste of the dates. By removing the color and taste, the resultant syrup may be a natural sweetener that is neutral in taste (e.g., usable to sweeten other food products where the distinct taste of dates is not preferable). An example of this may be mango juice, where mango concentrate may be combined with this neutral sweetener to offer a finished beverage with a desired level of sweetness without adding sugar (e.g., processed sugar and/or any date taste and/or any date color). Similarly, this may be utilized to make mango, grape, berry, and/or the like flavored gummies. Additionally or alternatively, this syrup may be used to make marshmallows that have a neutral sweetness, much like conventional marshmallows made from sugar. This use of an adsorbent resin in the processing of dates is unique, as no date syrup has heretofore been produced by removing the color and/or odor of the dates being processed, which may remove the essence of the material filtered.

At operation 170 of process 100, any suitable reverse osmosis operation(s) (e.g., any suitable mechanical process(es)) may be utilized to reduce the water content of the solution in order to optimize the usage of an evaporator (e.g., during a concentrating process for the remaining solution to prepare date sugar concentrate). Such reverse osmosis may be configured to remove some of the water to increase the concentration of date sugar in the remaining solution (e.g., to increase throughput of the evaporator (e.g., concentration of the juice into sugar)). Heretofore, no one has attempted to produce such date syrup utilizing any of these operations (e.g., ultrafiltration, resin decolorizing, and/or reverse osmosis). Instead, conventional date syrup may be darker and more viscous (e.g., higher in sugar concentration).

It is notable that each of the above operations, although arranged in-line, may remain vastly separate processes. If at the output of any stage, the solution (e.g., juice) is found to be failing any required or desired parameter(s) (e.g., degree of Brix (e.g., soluble sugars), color (e.g., per the International Commission for Uniform Methods of Sugar Analysis (“ICUMSA”)), and/or acidity (e.g., pH)), it may be cycled back to the beginning of that operation. The resin (e.g., of operation(s) 160) may be of the nature of an adsorbent resin. Process 100 may include a looping process where the solution may keep cycling through one or more resin vessels until the desired color and/or other desired characteristic(s) may be reached.

The operations shown in process 100 of FIG. 1 are only illustrative and existing operations may be modified or omitted, additional operations may be added, and the order of certain operations may be altered.

As shown in FIG. 2, an exemplary system 200 may be provided for preparing date sugar concentrate from dates (e.g., for use in any suitable forms and/or formulations (e.g., gummies, juices, marshmallows, etc.) of the disclosure), using any suitable process(es) (e.g., process 100 of FIG. 1). As shown, any suitable combiner subsystem(s) 202 may be used to combine any suitable material inputs, such as combiner material inputs 201a and 201b, to produce any suitable combiner material output 203. For example, combiner subsystem 202 may include any suitable crumbler and/or any other suitable machinery (e.g., on load cells, water mixing equipment, etc.) that may be operative to mix any suitable amount of combiner material input 201a with any suitable amount of combiner material input 201b to provide a new combined material output (e.g., solution) 203. Combiner material input 201a may be any suitable material(s), such as a supply of dates (e.g., 4.7 tons of dates per hour or up to 5 tons per hour), where the supply of dates may have any suitable characteristics, such as 18% or less stones, 18% or less moisture, and/or the like. Fruit from the Phoenix dactylifera (i.e., the date palm) may be cultivated and utilized as combiner material input 201a (e.g., any suitable species of wild date palms may be used). Combiner material input 201b may be any suitable material(s), such as a supply of water and/or other suitable solvent(s), where the supply of water or the like may have any suitable characteristics, such as 70° Celsius water added to obtain 15 Brix in a dissolving tank, where, for 1 ton of dates, 3.0 to 3.3 tons of water may be added to combiner subsystem 202 (e.g., as may be adjusted by any suitable programmable logic controller (“PLC”), such as a level transmitter subsystem (“LT”) 208), and/or the like. LT 208 may be any suitable level transmitter that may be configured to determine the amount of solution in the receiving tank (e.g., tank subsystem 210) below the destoners (e.g., pit removers), where the solution may be a combination of water, date fibers, and sugar extracted into the water. The water of combiner material input 201b may be clean water (e.g., from an RO subsystem), such as water with a total dissolved solids (“TDS”) rate of below 25 TDS. Any suitable pump subsystem 204 may be provided to pump combiner material output 203 from combiner subsystem 202 into any suitable destoner subsystem 206 as pumped combiner material output 205. Pump subsystem 204 may be controlled by a programmable logic controller that may be configured to factor for the levels of solution in the tank monitored by LT 208. In some embodiments, such a tank (e.g., tank subsystem 210) may be always maintained at levels allowing it to receive solution from the combiner subsystem 202 (e.g., via destoner subsystem 206), which may ensure workable operational fluidity. An enzyme may be used to breakdown the cellulose, which can allow the sugar from the dates to diffuse into the water within combiner subsystem 202. Citric acid may be optional and may depend on the quality of dates used, where the addition of citric acid may be used, in some embodiments, only when the pH of the raw dates used is above a certain threshold (e.g., 5). The enzymes used may be blends of cellulase and pectinase, which may be used to breakdown the cellulose to release the sugars better as well as breakdown the pectin so that the syrup may not gel due to the presence of natural pectin in dates.

Destoner subsystem 206 may include any suitable destoner and/or peel separator and/or any other suitable machinery that may be operative to remove the pits (e.g., seeds) and/or any other suitable element(s) (e.g., date flesh, water, etc.) from pumped combiner material output 205 to provide any suitable material output(s), such as a first destoner material output 207a and a second destoner material output 207b (e.g., through operation(s) 110 of process 100). For example, the pits and/or any other suitable material (e.g., hard material) may be removed (e.g., mechanically) through any suitable portion(s) of destoner subsystem 206 (e.g., a perforated stainless steel drum). The liquid, pits, and fiber may pass through destoner subsystem 206 (e.g., through a perforated drum), where the liquid and fiber of pumped combiner material output 205 may be pushed through destoner subsystem 206 (e.g., pushed through the perforations) as first destoner material output 207a (e.g., for receipt by any suitable target, such as a holding tank subsystem 210), while the pits and any other hard material of pumped combiner material output 205 (e.g., stones, peels, crowns, etc.) may be guided through a different exit of destoner subsystem 206 as second destoner material output 207b (e.g., for receipt by any suitable target, such as any suitable crusher subsystem 240 (e.g., for coarse crushing and/or any other suitable purpose(s))). In some embodiments, this may be done using two or more vats, where second destoner material output 207b may have any suitable characteristics, such as 13-14% moisture, 900 kilograms per hour (e.g., up to 18% moisture (e.g., residual moisture that may be in the pits and the moisture that may coat the pits)), and/or the like. The pits may come off clean without any notable fiber but they may be wet, and if they are crushed and left with the moisture on them they may rot. When coated in moisture, they may be dried then bagged to ensure that no rotting occurs.

Holding tank subsystem 210 may receive and hold first destoner material output 207a and provide any suitable portion of first destoner material output 207a from holding tank 210 to any suitable pump subsystem 212 as holding tank material output 211, which may be pumped out from pump subsystem 212 as pump material output 213 in any suitable manner, such as based on any suitable control signal(s) from any suitable controller, such as a speed controller subsystem (“SC”) 218. Pump material output 213 may be received from pump subsystem 212 by any suitable refractometer subsystem 214 that may be configured to measure an index of refraction (e.g., refractometry) of pump material output 213 (e.g., of liquid and fiber of pumped combiner material output 205) and provide pump material output 213 as refractory material output 215 for receipt by any suitable flow meter subsystem (“FM”) 216. Any suitable flow rate subsystem 220 may be provided to work with refractometer subsystem 214. Refractometer subsystem 214 may be configured to measure the degree of brix (e.g., the level of dissolved sugars in the solution). At this stage, a measurement of sugars in the solution may be measured and used to determine how much sugar has been extracted and how much is remaining in the fibers. This may provide an indication as to how long the enzymes may be working for before the fibers may be separated from the solution containing all of the sugars. FM 216 may be configured to determine the flow rate of refractory material output 215 from refractometer subsystem 214 in order to provide any suitable control signal(s) to SC 218 and to provide refractory material output 217, which may be any suitable portion or none or the entirety of refractory material output 215. A reading made by FM 216 may be used to determine the rotation speed of the motor (e.g., of pump 212), which may be controlled by SC 218. Refractory material output 217 may be a solution with a lot of suspended fiber so the pumping rates and flow rates may vary depending on the amount of fiber in the line. This fiber is usually not dissolved, so the flow may be weaker in pockets of higher rates of fiber, so the speed controller may be configured to compensate the flow using higher rotation speed.

Any suitable meter, such as any suitable acidity or basicity or potential of hydrogen (“PH”) meter subsystem 222, may be provided to measure the hydrogen-ion activity in refractory material output 217 (e.g., in the water-based solution of the liquid and fiber of pumped combiner material output 205), which may indicate its acidity or alkalinity expressed as pH. This pH reading of meter 222 may be used to control any suitable pump subsystem 226 to dictate the amount (if any) of any suitable citric acid 224 that may be pumped as a citric acid pump input 225 through pump 226 as a citric acid pump material output 227, where any such citric acid pump material output 227 may be combined with refractory material output 217 to provide a potentially citric refractory material output 217′ that may or may not include any suitable amount of citric acid 224 for potentially facilitating the work of any suitable enzymes (e.g., enzymes 228) that may be added to potentially citric refractory material output 217′. In some embodiments, the higher the pH above 4.8, the more prone the material may become to spoilage (e.g., yeast/bacterial activity). In some embodiments, the pH levels in the syrup should be below 5, such that citric acid may be dosed when needed to ensure that the product meets such specifications. The dosing mechanism may be done as follows: a solution of citric acid dissolved in water may be placed in the citric acid tank, which may be coupled to tank subsystem 232 (e.g., to all 8 holding (e.g., formulation) tanks), where such citric acid may be dosed into the tank(s) which may have a pH level higher than required using a small dosing pump.

Any suitable pump subsystem 230 may be provided to dictate the amount (if any) of any suitable enzymes 228 (e.g., enzymes that may be operative for breaking of proteins and/or pectin molecules (e.g., cellulose) of potentially citric refractory material output 217′) that may be pumped as an enzyme pump material input 229 through pump 230 as an enzyme pump material output 231, where any such enzyme pump material output 231 may be combined with potentially citric refractory material output 217′ to provide a potentially enzymed citric refractory material output 217″ that may or may not include any suitable amount of enzymes 228 and any suitable amount of citric acid 224 for potentially facilitating the work of any suitable enzymes (e.g., enzymes may be used to breakdown the cellulose, which can allow the sugar from the dates of material output 217′ to diffuse into the water of material output 217′ for providing material output 217″). The citric acid may be injected into the pipeline (e.g., without a mixer).

Any suitable target, such as a dissolving tank subsystem 232, may be provided to receive potentially enzymed citric refractory material output 217″ and to provide potentially broken down material output 233 to any suitable target, such as to any suitable pump subsystem 234 that may receive potentially broken down material output 233 and may pump it out as potentially broken down material output 235 in any suitable manner to any suitable target, such as any suitable filter-press subsystem 236. Pump subsystem 234 may include a positive displacement pump that may be configured to maintain the output flow even against a significant amount of back pressure. This is because it may be pumping the solution through compressed fine mesh plates that may collect the fibers and any sedimentation (e.g., above 50 microns). The back pressure may build up and conventional pumps may end up not moving liquid against this amount of back pressure. Dissolving tank subsystem 232 may include at least one dissolving tank that may be configured to receive and hold potentially enzymed citric refractory material output 217″ in combination with any other suitable inputs, such as a water material input 223 (e.g., water with a total dissolved solids (“TDS”) of less than 400 parts per million (“ppm”)). During a holding period within such a dissolving tank, pulp of potentially enzymed citric refractory material output 217″ (e.g., date pulp of combiner material input 201a that survives to subsystem 232 as part of material output 217″) may be chemically and/or physically disintegrated and saccharides may be slowly extracted from the pulp and diluted into liquid phase (e.g., Brix 15°, Temperature=48-52° Celsius, PH=4-4.8 may be set by any suitable PLC(s) (e.g., of subsystem 232), etc.). For example, the temperature may be important because the solution may contain live enzymes that have already done their job at optimal temperatures (e.g., of 40-50° Celsius) and now they may need to be denatured by the pasteurizing line at up to 90° Celsius. The enzymes may sit in the solution at an optimal temperature for about 40 minutes to one hour before the solution may be forwarded to the following section. In some embodiments, to promote an efficient use of system 200, dissolving tank subsystem 232 may include multiple such dissolving tanks, such as 8 dissolving tanks, where during any given time period (e.g., an hour), 2 of the tanks may be filled (e.g., with inputs 217″ and 223, another 2 of the tanks may be holding the materials for processing (e.g., pulp disintegration/extraction), another 2 of the tanks may be emptied (e.g., for pumping out output 233), and another 2 of the tanks may be cleaned, such that subsystem 232 may be continuously used for providing material output 233. Each tank of dissolving tank subsystem 232 may be any suitable size (e.g., a size for holding 6,000 liters of material).

Filter-press subsystem 236 may include any suitable filter press machinery and/or any other suitable machinery that may be operative to remove any suitable date flesh and/or soft material (e.g., calyxes or otherwise) of the dates of the solution of potentially broken down material output 235 received by subsystem 236 (e.g., to remove calyxes, fibers, date flesh, other soft material, and/or the like of the dates of combiner material input 201a that may survive to subsystem 236 as part of material output 235) and to provide any suitable material output(s), such as a first filter-press material output 237a and a second filter-press material output 237b (e.g., through operation(s) 120 of process 100). This may allow for physical separation between juice and pulp (e.g., protein and pectins). The pulp may be the date fibers that the filter may separate from the solution. The pectins may be removed by the enzymes. The saccharides may be now fully dissolved in the water that may be extracted from the filter press. For example, the fibers and/or any other suitable material (e.g., soft material (e.g., calyxes, fibers, date flesh, etc.)) may be removed (e.g., mechanically) from output 235 (e.g., from the date-sugar saturated water output by subsystem 232) through any suitable portion(s) of filter-press subsystem 236. The liquid and fiber and/or other soft materials may pass through filter-press subsystem 236, where the liquid of pumped potentially broken down material output 235 may be processed through filter-press subsystem 236 and provided as first filter-press material output 237a (e.g., juice extract for receipt by any suitable target, such as a pump subsystem 248, for further use in other process(es) (e.g., centrifugal separation, micron filtering, ultrafiltration, resin filtration, and/or the like (e.g., for providing date sugar concentrate))), while the fibers and/or any other soft material of pumped potentially broken down material output 235 (e.g., calyxes, fibers, date flesh, etc.) may be processed through filter-press subsystem 206 and provided as second filter-press material output 237b (e.g., date fibers after sugars extracted from the water, pulp reject (e.g., 3 Brix (e.g., 3% sugar left over in the pulp that was not extracted into the water (e.g., waste that may go into the animal feed with the pulp that has been dried)) for receipt by any suitable target, such as a mover subsystem 238, for further use in other process(es) (e.g., drying, grinding, and/or the like (e.g., for providing animal feed)), etc.), while the brix of the water exiting the filter press may be above 15.

Pump subsystem 248 may be configured to receive first filter-press material output 237a and pump first filter-press material output 237a out as first filter-press material output 249 in any suitable manner to any suitable target, such as any suitable separator subsystem 262. A pump of pump subsystem 248 may be manually controlled to pump out the collected water containing the extracted sugar.

Separator subsystem 262 (e.g., a centrifugal separator subsystem) may be provided that may include any suitable machinery that may be operative to separate out any sludge from first filter-press material output 237a or first filter-press material output 249, as may be received by separator subsystem 262 from filter-press subsystem 236 or pump subsystem 248, and to provide any suitable material output(s), such as a first separator material output 263a and a second separator material output 263b (e.g., through operation(s) 140 of process 100). For example, separator subsystem 262 may include any suitable machinery that may be utilized to spin the solution of first filter-press material output 249 at very high speeds and rely on centrifugal force to pull the heavier materials of first filter-press material output 249 (e.g., contaminants, sludge, etc.) of the solution to an outer rim of the machinery where they may be captured and discarded as second separator material output 263b while the lighter (e.g., clean) juice of the solution of first filter-press material output 249 may be retained (e.g., by flowing through a center of the separator) and then passed on by separator subsystem 262 as first separator material output 263a. The machinery may be spun at auto intervals, which may depend on the amount of second separator material output 263b being centrifuged. For example, separator subsystem 262 may include conical plates that spin at very high speeds and push out the particles that are heavier (e.g., higher densities (e.g., larger)). These particles may be pushed out to the sides and accumulate at the top of the spin axis, where the clarified material may flow down the center (e.g., similar to a drain).

Any suitable subsystem, such as any suitable turbidity (“TD”) meter 264, may be provided to analyze separator material output 263a. For example, TD 264 may be configured to measure the turbidity of the filtered solution (e.g., a turbid solution is cloudy or murky solution). This may be used because centrifugal separators (e.g., of subsystem 262) may be used to reduce the turbidity of the solution. If the turbidity is higher than a certain threshold (e.g., 100 Nephelometric Turbidity Units (“NTUs”) or 170 NTUs or the like), an electronic valve (“EV”) 266 may be configured to recycle the solution back to go through the separators (e.g., centrifuges) and/or filter-press subsystem 236 again until the desired NTU is reached. This threshold may relate to the expected cycle times of the ultrafiltration (e.g., the higher the turbidity, the faster the ultrafiltration membranes may become clogged and require cleaning). TD 264 may be operative to pass separator material output 263a on to any suitable subsystem, such as EV 266, as TD separator material output 265. EV 266 may be operative to receive TD separator material output 265 and provide a first portion or none or the entirety of TD separator material output 265 as EV separator output 267a and/or a second portion or none or the entirety of TD separator material output 265 as TD separator recycled output 267b. Moreover, any suitable analysis of separator material output 263a by TD 264 may be used to provide any suitable control signal(s) from TD 264 to EV 266 for controlling how EV 266 may function for determining the amount (if any) of TD separator material output 265 that may be passed from EV 266 as EV separator output 267a and/or for determining the amount (if any) of TD separator material output 265 that may be passed from EV 266 as TD separator recycled output 267b. For example, TD 264 and EV 266 may be configured to pass any portion of TD separator material output 265 with a turbidity (e.g., haze) below a certain threshold (e.g., below 170 Nephelometric Turbidity Units (“NTUs”)) along as EV separator output 267a (e.g., for further processing to generate a final date sugar concentrate (e.g., to a holding tank subsystem 250)) and/or to pass any portion of TD separator material output 265 with a turbidity (e.g., haze) above a certain threshold (e.g., above 170 NTUs) along as TD separator recycled output 267b (e.g., in a loop recycle back for further earlier processing, such as that of filter-press subsystem 236 and/or separator subsystem 262 (e.g., to dissolving tank subsystem 232)). Therefore, TD 264 may be configured to measure the turbidity of output 263a of separator subsystem 262 and, if acceptable, EV 266 may pass it along as EV separator output 267a, otherwise EV 266 may recycle the material back to separator subsystem 262 (e.g., via subsystem 232 and/or via subsystem 236 or directly to subsystem 262). Therefore, TD 264 may be configured as a checking subsystem to ensure that separator subsystem 262 did its job correctly and, if not, TD 264 may have separator subsystem 262 re-do the job. A turbidity threshold of TD 264 and EV 266 may be variable based on the dates used (e.g., at material 201a) and/or any desired results. Preferably, the threshold for TD 264 may be 100 NTUs but ought to be at least less than 200 NTUs. In some embodiments, 170 NTUs may be used. The lower the threshold, the cleaner the product, while the higher the threshold, sometimes the better level of flow.

Holding tank subsystem 250 may be any suitable subsystem that may be configured to receive and hold EV separator output 267a and provide any suitable portion of EV separator output 267a from holding tank 250 to any suitable pump subsystem 252 as holding tank material output 251, which may be pumped out from pump subsystem 252 as pump material output 253 in any suitable manner. A pump of pump subsystem 252 may be an electric motor controlled pump to forward the juice through a pasteurizer.

Pump material output 253 may be received from pump subsystem 252 by any suitable pasteurizer subsystem 259 that may be configured to inactivate any suitable enzymes of pump material output 253. For example, pasteurizer subsystem 259 may include a heater subsystem 254 that may be operative to receive pump material output 253 and provide heater material output 255 (e.g., pump material output 253 as heated by any suitable amount by heater subsystem 254 (e.g., 40-90° Celsius)). Pasteurizer subsystem 259 may also include any suitable valve subsystem (“EV”) 256 that may be operative to receive heater material output 255 and provide a first portion or none or the entirety of heater material output 255 as pasteurizer separator output 257a and/or a second portion or none or the entirety of heater material output 255 as pasteurizer recycled output 257b. Moreover, pasteurizer subsystem 259 may include any suitable temperature sensor or temperature transmitter subsystem (“TT”) 258 that may be operative to determine the temperature of heater material output 255 in order to provide any suitable control signal(s) to EV 256 for controlling how EV 256 may function for providing pasteurizer separator output 257a and/or pasteurizer recycled output 257b. If the liquid reaches a desired temperature (e.g., as measured by TT 258), then EV 256 may be configured to release the liquid to cooler subsystem 260). However, if the liquid has not reached a desired temperature (e.g., a temperature level suitable to ensure efficient pasteurization), then EV 256 may be configured to recycle the liquid back to heater subsystem 254. For example, in some embodiments, heater subsystem 254 may receive any suitable pasteurizer recycled output 257b from any suitable pasteurizer subsystem 259 of system 200, which may be used by heater subsystem 254 in any suitable manner. If the liquid does not reach the desired temperature, it may be recycled back through a tubular heat exchanger of heater subsystem 254 to reach the target pasteurizing temperature. Once it reaches that temperature, it may then be cooled and released through the electronic valve. For example, EV 256 may be a three-way valve. Pasteurizer subsystem 259 may also include any suitable cooler subsystem 260 that may be operative to receive pasteurizer separator output 257a from EV 256 and provide cooler material output 261 (e.g., pasteurizer separator output 257a as cooled by any suitable amount by cooler subsystem 260 (e.g., 90-40° Celsius)). For example, pasteurization may occur at 90° Celsius. Therefore, the liquid may go through heater subsystem 254 (e.g., heating tubes thereof) until the temperature of the liquid reaches 90° Celsius. The liquid may then be released to the following section where it may be cooled back to 40° Celsius (e.g., by cooler subsystem 260). The temperature ranges may be set on one or more controller(s) so that the system does not reject liquid (e.g., liquid with a temperature higher than a target temperature (e.g., 90° Celsius)). This type of analog system may be designed to operate within set parameters to minimize the margins for error.

Cooler material output 261 may be provided by pasteurizer subsystem 259 to any suitable target, such as any suitable micronfiltration subsystem 1240, that may include any suitable machinery (e.g., a pass through filter) that may be operative to filter any particles from the solution of cooler material output 261 larger than a certain threshold, such as 1 micron (e.g., through operation(s) 130 of process 100). This threshold may be set to alleviate the job that may be carried out by a later filtration subsystem (e.g., an ultrafiltration subsystem 280), which may be designed to catch particles smaller than this threshold (e.g., particles smaller than 1 micron). Any material of cooler material output 261 that may be filtered out by micronfiltration subsystem 1240 (e.g., suspended date fibers less than 1 micron in size) may be removed as waste (e.g., when replacing any suitable micronfilter(s) of micronfiltration subsystem 1240), while the remaining solution of cooler material output 261 may be passed along by micronfiltration subsystem 1240 as micronfiltration material output 1241, which may be received by any suitable target, such as a buffer tank subsystem 268.

Buffer tank subsystem 268 may be any suitable subsystem that may be configured to receive and hold micronfiltration material output 1241 and provide any suitable portion or none or the entirety of micronfiltration material output 1241 as buffer tank material output 269 from buffer tank subsystem 268 to any suitable pump subsystem 270, and buffer tank material output 269 may be pumped out from pump subsystem 270 as pump material output 271 in any suitable manner, such as based on any suitable control signal(s) from any suitable controller, such as a level transmitter subsystem (“LT”) 276 (e.g., to regulate downstream flow). Pump material output 271 may be received from pump subsystem 270 by any suitable pump subsystem 272, and pump material output 271 may be pumped out from pump subsystem 272 as pump material output 273 in any suitable manner, such as based on any suitable control signal(s) from any suitable controller, such as a speed controller subsystem (“SC”) 274, to any suitable target, such as an ultrafiltration subsystem 280. Any suitable pressure transmitter subsystem (“PT”) 275 may be provided to determine the pressure of pump material output 273 from pump subsystem 272 in order to provide any suitable control signal(s) to SC 274 and/or to LT 276. This portion of system 200 may begin by ensuring that there is a continuous flow of liquid through the ultrafiltration unit(s) of ultrafiltration subsystem 280, which may be accomplished by regulating the speed in which the liquid is pumped into the unit(s) by using SC 274 (e.g., a pump motor speed controller). LT 276 associated with tank subsystem 268 may be configured to ensure that the liquid level in the tank may be continuously monitored and that, if the tank is emptied, a pump of subsystem 270 may be stopped, which may help ensure a continuous flow of liquid throughout the system. Pump subsystem 270 may be configured to send the liquid to pump subsystem 272 at a single constant speed, where pumps of subsystems 270 and 272 may be separated by any suitable distance (e.g., about 100 meters worth of pipes away from one another). Pump 272 may receive the liquid and pump it at any suitable desired speed.

Any suitable meter or transmitter subsystem, such as any suitable flow transmitter (“FT”) 278, may be provided to determine the flow rate of pump material output 273 from pump subsystem 272 and then pass pump material output 273 as FT pump material output 279 to any suitable target, such as ultrafiltration subsystem 280. FT pump material output 279 may be any suitable temperature (e.g., cooled to 40-45° Celsius) and/or any suitable color (e.g., 3,000-16,000 international units (IUs), which may be reduced by subsystem 280 and again by subsystem 298). Ultrafiltration subsystem 280 may include any suitable filter machinery (e.g., with a network of membrane fitted tubing) and/or any other suitable machinery that may be operative to remove micron-sized particles from the solution of FT pump material output 279 received by subsystem 280. Such ultrafiltration may be configured to remove any suspended non-soluble solids that are too small to be filtered out by filter-press subsystem 236 (e.g., by operation(s) 120 of FIG. 1). For example, such ultrafiltration conducted by ultrafiltration subsystem 280 may include one or more subprocesses wherein sub-micron particles may be filtered out, and wherein, in some embodiments, even some of the mineral content naturally found in dates may be removed. Therefore, ultrafiltration subsystem 280 may be utilized during the processing of dates (e.g., of combiner material input 201a) for the purpose of decolorizing and/or de-odorizing the resulting syrup (e.g., through operation(s) 150 of process 100). For example, retentate waste (e.g., material greater than a certain size (e.g., 10 micrometers), such as macromolecules, big proteins, vitamins, mineral salts, and/or the like) may be removed (e.g., mechanically) from FT pump material output 279 (e.g., from the date-sugar saturated water output by subsystem 268 via pumps 270 and 272 and FT 278) through any suitable portion(s) of ultrafiltration subsystem 280. The liquid and other permeate (e.g., material less than a certain size (e.g., 10 micrometers)) may pass through ultrafiltration subsystem 280. Therefore, the liquid and permeate of FT pump material output 279 may be processed through ultrafiltration subsystem 280 and provided as a first ultrafiltration material output 281a (e.g., permeate (e.g., juice) for receipt by any suitable target, such as a color meter (“CL”) or any other suitable subsystem 288, for further use in other process(es) (e.g., resin filtration, and/or the like (e.g., for providing date sugar concentrate))), while the retentate waste material of FT pump material output 279 may be processed through ultrafiltration subsystem 280 and provided as second ultrafiltration material output 281b (e.g., retentate waste for receipt by any suitable target, such as an FT 282, for further use in other process(es)). Any suitable meter or transmitter subsystem, such as any suitable flow transmitter (“FT”) 282, may be provided to determine the flow rate of second ultrafiltration material output 281b from ultrafiltration subsystem 280 and then pass second ultrafiltration material output 281b as FT pump material output 283 to any suitable target subsystem, such as any suitable flow rate subsystem 286. Any suitable subsystem, such as any suitable flow indicator (“FICA”) 284 may be configured to receive any suitable flow rate determination(s) from FT 282 and/or FT 278 and process the same for generating any suitable control signal(s), which may be provided to flow rate subsystem 286 for controlling the flow of FT pump material output 283 from flow rate subsystem 286 as flow rate pump material output 285. Therefore, ultrafiltration subsystem 280 may have one inlet (e.g., to receive juice (e.g., FT pump material output 279)) and a first outlet (e.g., to provide permeate (e.g., first ultrafiltration material output 281a (e.g., what goes through the filtration membranes and is filtered and proceeds to a later resin section (e.g., subsystem 298)))) and a second outlet (e.g., to provide retentate (e.g., second ultrafiltration material output 281b (e.g., the liquid that did not go through the membranes (e.g., unfiltered liquid) that may be cycled back into earlier stages to re-do certain subprocesses))). FTs 278 and 282 may be configured to determine the parameters of flow 279 and 281b, where a predetermined ratio may be preferred for input into the system (e.g., 5.8% retentate), whereby, if there is 17 cubic meters per hour of FT pump material output 279 going into ultrafiltration subsystem 280 and an open outlet to everything that goes through the membrane (e.g., permeate), the system may be configured to factor for about 1 cubic meter to go out of the retentate line through a valve fitted with an actuator 286 that may open in varying degrees to let out a measured 1 cubic meter per hour worth of flow. Each inlet and outlet may be fitted with a flow meter to determine the performance of this unit. FICA 284 may be an alarm unit to indicate issues related to flow and may be configured to instigate fixes. For example, in some embodiments, subsystem 280 may include 2 units with 96 membranes each. If high pressure is used, then the membranes may be configured to block flow below a set value and then switch over, and a small amount of water may be re-circulated through the last set of membranes in order to dilute the juice and thus to recover more sugar (e.g., 12-16 hours of filtration time per cycle, shelf life 700 cycle, where juice may be permeate that passes through membrane (e.g., AT 7-8 Bar)). Retentate waste of second ultrafiltration material output 281b from ultrafiltration subsystem 280 may include material(s) larger than a certain filtration threshold (e.g., 10 micrometers), such as 1000-1400 kilograms per hour, 15% moisture contenat, macromolecules, big proteins, vitamins, mineral salts, and/or the like (e.g., this retentate may be returned to earlier subprocesses (e.g., to be added to freshly hydrated dates) in some embodiments or discarded), while permeate of first ultrafiltration material output 281a from ultrafiltration subsystem 280 may include material(s) smaller than a certain filtration threshold (e.g., 10 micrometers (e.g., the size of particles that may pass through these membranes (e.g., particles suspended in the liquid sized smaller than 10 micrometers may be passed for further processing, while all larger than this threshold may never pass to the next stage and may be considered waste))).

Any suitable color meter (“CL”) subsystem 288 may be configured to receive first ultrafiltration material output 281a from ultrafiltration subsystem 280 to perform certain analysis on first ultrafiltration material output 281a and provide CL ultrafiltration material output 289. CL 288 may include any suitable color meter that may determine a quantified color of the analyzed material (e.g., material 281a may be determined to have a color of 1,500-3,000 IU (e.g., a clearer color than material 279). Any suitable subsystem, such as a flow meter (“FM”) 290, may be configured to receive CL ultrafiltration material output 289 and any suitable other material input 289a (e.g., to reject liquid at any suitable rate (e.g., about 6% based on a liquid flow rate of 17 cubic meters per hour)), where FM 290 may be configured to utilize CL ultrafiltration material output 289 and any material input 289a to provide any suitable FM ultrafiltration material output 291 to any suitable target subsystem, such as a holding tank subsystem 292. FM 290 may include any suitable flow meter that may be configured to measure the output of liquid that passed through the ultrafiltration (e.g., material of output 281a/289). The sum of FM 290 and FT 282 should be equal to the inlet flow of FT 278. The measurements of FT 282 and F 278 may be calculated by the controller and FICA 284 may be configured to influence how wide the reject valve 286 opens to let out 1,000-1,400 Kg/hr or any other suitable flow (e.g., no more than 14,00 Kg per hour because of the size limit of that pipe, which may be much smaller than the inlet pipe (e.g., 4 inch pipe) leading into the ultrafiltration subsystem in some embodiments).

Holding tank subsystem 292 may be any suitable subsystem that may be configured to receive and hold FM ultrafiltration material output 291 and provide any suitable holding tank material output 293 from holding tank subsystem 292 to any suitable pump subsystem 294, and holding tank material output 293 may be pumped out from pump subsystem 294 as pump material output 295 in any suitable manner, such as based on any suitable control signal(s) from any suitable controller, such as a level transmitter subsystem (“LT”) 296 (e.g., to regulate downstream flow), while such pump material output 295 may be received from pump subsystem 294 by any suitable resin filtration subsystem 298 (e.g., system 200 may be configured such that pump material output 295 may be about 15° Brix (e.g., about 15% sugar in the solution), flowing at about 17,000-19,200 kilograms/hour (e.g., 17-19.2 cubic meters of liquid per hour), and with a color of about 1,500-3,000 IU). Additionally or alternatively, in some embodiments, holding tank subsystem 292 may receive any suitable decolorized material output 1207b from any suitable subsystem, such as EV 1206 or otherwise of system 200, which may be used by holding tank subsystem 292 in any suitable manner. If the liquid does not get decolorized from about 1,500-3,000 IU (e.g., of output 295) down to about 150 IU or below (e.g., at output 299a/1203/1205 of resin filtration subsystem 298), an EV 1206 may be configured to send the liquid back to holding tank 292 as output 1207b so that it may do another pass through the resin section.

Resin filtration subsystem 298 may include any suitable filter machinery (e.g., with an adsorbent resin (e.g., Dowex Optipore SD-2)) and/or any other suitable machinery that may be operative to decolorize and/or vastly reduce the turbidity of the solution of pump material output 295 received by subsystem 298. Subsystem 298 may be configured to reduce the turbidity, as may separator subsystem 262 and/or ultrafiltration subsystem 280. The turbidity of liquid may enter subsystem 298 at about 100-150 NTU and may be reduced to less than 15 NTUs (e.g., to about 10-12 NTUs (e.g., from lightly cloudy to polished and very clear)). Such resin filtration may be configured to remove the minerals that give date juice its date taste and aroma, such that processing by subsystem 298 (e.g., operation(s) 160 of FIG. 1) may result largely in the removal of the natural color and/or taste of the dates from the date sugar concentrate being produced. For example, such resin filtration conducted by resin filtration subsystem 298 may include one or more subprocesses wherein color and/or pectins may be removed chemically and/or physically, such as through use of resin operative to absorb 80,000 IU/Lit with 70% efficiency, which may have shelf life of 5,00 cycles. For example, the liquid before going into the resin may have about 1,000-1,500 IU worth of color (e.g., looks like 50/50 apple juice and water) and once it goes out of the resin section it may be reduced to about 150 IU or less. This may be measured by liter (e.g., every liter may lose about 1,350 IU into the resin), where each liter of resin can absorb up to 80,000 before it may need to be washed. The total IUs leaving the ultrafiltration and the total IUs leaving the resin section may be measured, while the number of liters of resin in the tank may be known, to calculate when the resin may need to be washed to start the next cycle. Therefore, the resin filtered liquid of pump material output 295 may be processed through resin filtration subsystem 298 and provided as a first resin filtration material output 299a (e.g., decolorized and/or low turbidity juice for receipt by any suitable target subsystem, such as a PH meter 1202 or any other suitable subsystem, for further use in other process(es) (e.g., for providing date sugar concentrate)). The flow of first resin filtration material output 299a may be about 19,200 kilograms per hour, while any waste material of pump material output 295 may be processed through resin filtration subsystem 298 and provided as second resin filtration material output 299b (e.g., rejected liquid, which may be recycled back to any earlier subprocess (e.g., goes back to the beginning of the process to be mixed with fresh dates), such that system 200 may support a continuous cycle that never ends (e.g., only a few tons of waste may be realized when ultimate shut down may be reached for prolonged maintenance after weeks of running continuously 24 hours per day)). For example, in some embodiments, subsystem 298 may include 4 decolorization vessels, 5,000 L resin each (e.g., 2 vessels running 10 hours, then 4 hours regeneration), top up 3% each 100 cycles (3,000 lt/yr) cycles. In some embodiments, there may be two machines of everything so that one machine may be washed while the other may be run (e.g., to achieve continuous production). Each resin section may include 2 tanks, where each tank may contain 5,000 liters of resin and the liquid may flow through the resin entering from the bottom and exiting from the top (e.g., a first pass goes through the first tank and the second pass goes through the second tank (e.g., if after going through the second tank the color is about 150 IU, the liquid may return to the holding tank in preparation to go through the system again)). By removing the color and taste through such resin filtration, the resultant syrup may be a natural sweetener that is neutral in taste (e.g., usable to sweeten other food products where the distinct taste of dates is not preferable). An example of this may be mango juice, where mango concentrate may be combined with this neutral sweetener to offer a finished beverage with a desired level of sweetness without adding sugar (e.g., processed sugar and/or any date taste and/or any date color). Similarly, this may be utilized to make mango, grape, berry, and/or the like flavored gummies. Additionally or alternatively, this syrup may be used to make marshmallows that have a neutral sweetness, much like conventional marshmallows made from sugar. This use of an adsorbent resin in the processing of dates is unique, as no date syrup has heretofore been produced by removing the color and/or odor of the dates being processed, which may remove the essence of the material filtered.

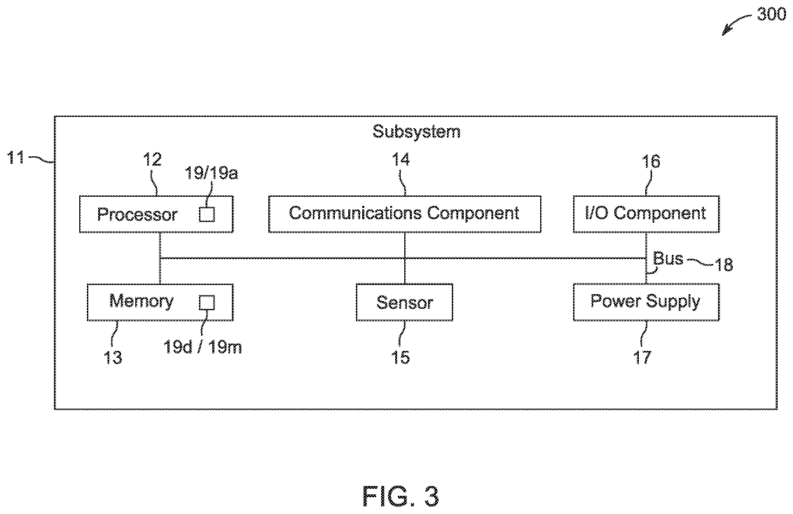

First resin filtration material output 299a may be received by any suitable subsystem, such as PH 1202, which may analyze first resin filtration material output 299a in any suitable manner and provide PH material output 1203. Here, for example, citric acid may be injected into the line of liquid, before it is concentrated, the pH meter may measure to make sure the pH is within range, and only if it is higher than 5 may the citric acid solution be injected into the line. PH material output 1203 may be received by any suitable subsystem, such as CL 1204, which may analyze PH material output 1203 in any suitable manner and provide CL material output 1205. The color meter may be configured to read the color value and if higher than 150 IU recycles the material back to holding tank 292 through EV 1206 (e.g., a three-way valve). CL material output 1205 may be received by any suitable subsystem, such as EV 1206, which may analyze CL material output 1205 in any suitable manner and provide a first portion or none or the entirety of CL material output 1205 as a first EV material output 1207a (e.g., to be provided to any suitable target subsystem, such as a holding tank subsystem 1208) and/or provide a second portion or none or the entirety of CL material output 1205 as a second EV material output (e.g., decolorized material output) 1207b (e.g., to be provided to any suitable target subsystem, such as a holding tank subsystem 292). For example, EV 1206 may be configured to pass any CL material output 1205 with a color below a certain threshold (e.g., below 150 IU) along as first EV material output 1207a (e.g., for further processing to generate a final date sugar concentrate) and/or to pass any CL material output 1205 with a color above a certain threshold (e.g., above 150 IU) along as second EV material output 1207b (e.g., in a loop recycle back to holding tank 292). For example, a color meter of CL 1204 may be configured to determine the color of resin filtration material output 299a and communicate with any suitable controller (e.g., programable logic controller (“PLC”)) of system 200, where the color determination may determine the PLC reaction (e.g., if the reading is acceptable, the liquid may be allowed to pass (e.g., as material 1207a), but, if not, the liquid may be rejected (e.g., as material 1207b)). Any one, some, or all field instruments of system 200 (e.g., one, some, or each, FM, FT, LT, TT, CL, and/or the like) may be electrically or otherwise communicatively coupled to one or more controllers or PLCs of the system, each of which may have an operator interface to set parameters, monitor processes, and/or operate the machinery in manipulating manual values where required amongst other things (e.g., as described with respect to FIG. 3).

Holding tank subsystem 1208 may be any suitable subsystem that may be configured to receive and hold first EV material output 1207a and provide any suitable holding tank material output 1209 from holding tank subsystem 1208 to any suitable pump subsystem 1210, and holding tank material output 1209 may be pumped out from pump subsystem 1210 as pump material output 1211 in any suitable manner, such as based on any suitable control signal(s) from any suitable controller, such as a level transmitter subsystem (“LT”) 1212 (e.g., to regulate downstream flow), while such pump material output 1211 may be received from pump subsystem 1210 by any suitable nanofiltration subsystem 1214. The liquid in a tank of tank subsystem 1208 may have about 15% sugar dissolved in it (e.g., 15° Brix). A pump of pump subsystem 1210 may be a high pressure pump that may be coupled to a smart variable speed motor that may be designed to work within a range of power settings (e.g., based on a level read by LT 1212 (e.g., the lighter the tank level, the harder the pump may be running but never below a certain power and never above a certain power unless zero (e.g., a rule where power may be voltage)).

Nanofiltration subsystem 1214 (e.g., a reverse osmosis (“RO”) subsystem) may include any suitable filter machinery (e.g., with one or two or more sets of membranes (e.g., 18 membranes per set), such as with a cycle duration of 20 hours and/or shelf life 1,100 cycles) and/or any other suitable machinery that may be operative to filter the solution of pump material output 1211 received by subsystem 1214. Nanofiltration subsystem 1214 may be configured to filter out water. As an ultimate goal may be to concentrate the liquid, concentration may use heat but heat comes at a cost. A lot of water may be added in order to efficiently dilute the sugars into the water and be able to filter out the color without the use of chemicals. To optimize efficiency and increase productivity, system 200 may filter out the water (e.g., at subsystem 1214) to reduce the amount of labor that evaporators may have to do (e.g., in evaporating the water where the sugars and a small amount of water may remain). Subsystem 1214 may be designed where anything that goes through the membranes may be pure water (e.g., output 1215b) and that may be returned into the process (e.g., to mix with raw dates) while the liquid that does not pass through the membranes (e.g., output 1215a) may be sent to be concentrated. The liquid of pump material output 1211 may be processed through nanofiltration subsystem 1214 and provided as a first nanofiltration material output 1215a (e.g., a 22° Brix retentate for receipt by any suitable subsystem(s) for further processing to generate a final date sugar concentrate). When some pure water is removed from the solution, the concentration of sugars may increase, even slightly (e.g., at this stage it may grow to about 22% (e.g., 22° Brix), not because more sugar may be added but rather the carrying media (e.g., water) may be partially removed). Any suitable water may be removed from pump material output 1211 as processed through nanofiltration subsystem 1214 and provided as second nanofiltration material output 1215b (e.g., permeate demi water waste (e.g., TDS less than 10 mg/liter) for receipt by any suitable target for any further process(es)). This may include trace amounts of sugars. As fresh water may be continuously added to be mixed in with raw dates, this water may go back to the first stage of production. Therefore, nanofiltration subsystem 1214 may be utilized to reduce the water content of the solution of pump material output 1211 in order to optimize the usage of an evaporator (e.g., during a concentrating process for the remaining solution to prepare date sugar concentrate). Such reverse osmosis may be configured to remove some of the water to increase the concentration of date sugar in the remaining solution of first nanofiltration material output 1215a that may be provided to any suitable target, such as an evaporator subsystem 1218 (e.g., to increase throughput of an evaporator (e.g., concentration of the juice into sugar)).

Any suitable subsystem, such as an evaporator subsystem 1218 (e.g., a falling film evaporator (e.g., three-phase evaporator) subsystem), may be provided to receive and further process the solution of first nanofiltration material output 1215a. Evaporator subsystem 1218 may include any suitable machinery (e.g., one or more semi-permeable membranes for separating water molecules from other substances within first nanofiltration material output 1215a) and/or any other suitable machinery that may be operative to filter the solution of first nanofiltration material output 1215a received by evaporator subsystem 1218. First nanofiltration material output 1215a (e.g., 22° Brix, with a color of 160-200 IU) may be processed (e.g., at 12,000-13,200 kilograms per hour) through evaporator subsystem 1218 for providing a first evaporator material output 1221a (e.g., a 75° Brix, 50° Celsius syrup for receipt by any suitable subsystem(s) for further processing to generate a final date sugar concentrate) and a second evaporator material output 1221b (e.g., evaporated water (e.g., 7.7-10.0 tons per hour)). For example, evaporator subsystem 1218 may include one or more evaporator stages, such as a first stage evaporator subsystem 1218a that may receive and process first nanofiltration material output 1215a (e.g., 22° Brix, with a color of 160-200 IU) for providing a first stage evaporator material output 1217 (e.g., 28° Brix) using any suitable first stage evaporator characteristics (e.g., a max temperature of about 78° Celsius and/or a vac of 0.45 bar), a second stage evaporator subsystem 1218b that may receive and process first stage evaporator material output 1217 (e.g., 28° Brix) for providing a second stage evaporator material output 1219 (e.g., 42° Brix) using any suitable second stage evaporator characteristics (e.g., a max temperature of about 66° Celsius and/or a vac of 0.27 bar), and a third stage evaporator subsystem 1218c that may receive and process second stage evaporator material output 1219 (e.g., 42° Brix) for providing a third stage evaporator material output or first evaporator material output 1221a (e.g., 75° Brix) using any suitable third stage evaporator characteristics (e.g., a max temperature of about 50° Celsius and/or a vac of 0.13 bar). Therefore, for example, the Brix level of liquid entering evaporator subsystem 1218 (e.g., nanofiltration material output 1215a) may be about 22% and, by the time its evaporated, at about 70° Celsius in stage 1 it may be increased to 30-40%, then it may go to stage 2 at about 65° Celsius where it may be further concentrated to up to 60%, then it may go to stage 3 at about 55° Celsius where it may be further concentrated to about 75%, where it may be syrup at this stage. This may all happen under vacuum where the boiling point may be reduced significantly, and rapid evaporation may be achieved without damaging the product with high temperatures. The evaporated water (e.g., second evaporator material output 1221b) may be condensed from steam to pure water and may be returned to an earlier stage of the process (e.g., to a first stage of production to be mixed with raw dates (e.g., with material input 201a)). In some embodiments, steam may be directly fed into stage 1 and flow to stage 2 and flow to stage 3 from there, whereby it may be its hottest in stage 1 and may gradually get cooler by stage 3, and whereby the remaining steam and condensed water may be collected and returned (e.g., to the boiler to be reheated into steam again). Meanwhile, the sugar liquid may be circulated in closed pipes going through the steam area in stage 1 for heating the liquid and concentrating it through the process of evaporation. After being concentrated to a preset degree, it may be transferred to stage 2 and so on. Once it is transferred to the following stage, fresh liquid may take its place.

Any suitable pump subsystem 1221 may be configured to receive first evaporator material output 1221a and pump first evaporator material output 1221a out as evaporator material output 1223 in any suitable manner to any suitable target, such as any suitable buffer tank subsystem 1224. A refractometer (not shown) may be fitted on or adjacent third stage evaporator subsystem 1218c, whereby, during circulation, the refractometer may be configured to read the concentration of sugar in the liquid. When it is determined to reach 75%, the circulation pump may be stopped by any suitable control based on the refractometer determination and a valve may be opened and the syrup maybe forwarded by a pump of pump subsystem 1222 to transfer the finished syrup to a buffer tank of subsystem 1224. Buffer tank subsystem 1224 may receive and hold evaporator material output 1223 and provide any suitable portion or all of evaporator material output 1223 from buffer tank 1224 to any suitable pump subsystem 1226 as buffer tank material output 1225, which may be pumped out from pump subsystem 1226 as pump material output 1227 in any suitable manner to any suitable target, such as a heat sterilization subsystem 1228. In some embodiments, a pump of pump subsystem 1226 may be manually triggered when each stage 3 load of finished syrup is loaded into tank subsystem 1224.

For example, heat sterilization subsystem 1228 may include a heater subsystem 1230 that may be operative to receive pump material output 1227 and provide first heater material output 1231a (e.g., pump material output 1227 as heated by any suitable amount by heater subsystem 1230 (e.g., 95° Celsius for 120 seconds)). Heater subsystem 1230 may recycle any suitable hot water therethrough to enable such heating (e.g., using a hot water input 1227a and a hot water output 1231b). Heat sterilization subsystem 1228 may also include any suitable cooler subsystem 1232 that may be operative to receive provide first heater material output 1231a from heater subsystem 1230 and provide a first cooler material output 1233a (e.g., first heater material output 1231a as cooled by any suitable amount by cooler subsystem 1232 (e.g., 10° Celsius)). Cooler subsystem 1232 may recycle any suitable chilled water therethrough to enable such cooling (e.g., using a chilled water input 1231c and a chilled water output 1233b). Heat sterilization subsystem 1228 may include a tubular sterilizer, where the material may circulate in heater subsystem 1230 (e.g., pipes may be encased in a steam chamber) until the material reaches about 90° Celsius (e.g., in a range of 85-95° Celsius) for a total of 120 seconds, which may kill off any bacteria, yeast, and/or mold that might be in the syrup, after which, the material may be forwarded to circulate in cooler subsystem 1232 (e.g., in a cooled piping section), where the material may be rapidly cooled down to 10-20° Celsius. While pump material output 1227 and cooler material output 1233a may each be 75 Brix, cooler material output 1233a may have no turbidity and its color may be less than 250 ICUMSA and may be provided by heat sterilization subsystem 1228 at any suitable rate (e.g., 3200 kg/hr). The syrup may exit the evaporator as a syrup where the turbidity and color may have already diminished during the previous filtration processes. The evaporator may concentrate the decolorized juice that has almost no turbidity. This concentration may happen by evaporating the water under vacuum.

Cooler material output 1233a from heat sterilization subsystem 1228 may then be received and held by any suitable refrigeration subsystem 1234 for any suitable duration and environment for providing a refrigerated material output 1235. For example, refrigeration subsystem 1234 may include one or more jacketed refrigerated tanks (e.g., 16 tanks, each of which may be 50 cubic meters and 70 tons, and chilled at 7° Celsius plus or minus 2° Celsius), which may enable the product to be stored chilled.

Refrigerated material output 1235 from refrigeration subsystem 1234 may then be received and held by any suitable filling subsystem 1236 for any suitable duration and environment for providing a drum filled material output 1237. For example, filling subsystem 1236 may be any suitable aseptic filling machine for providing the date syrup into one or more drums (e.g., 40 drums may be filled per hour, with each drum being 280 kgs, where 4 drums may go on a single pallet (e.g., one by one with pallet rotation 360 degrees), and a nitrogen (N2) blanket may be provided in a bag before aseptically sealing it with a cap (e.g., to avoid oxidation and minimize discoloration). For example, nitrogen gas is heavier than oxygen so the nitrogen blanket may force out the oxygen in order to prevent any oxidation and darkening in color over time.

Drum filled material output 1237 from filling subsystem 1236 may then be received and held by any suitable cold room subsystem 1238 for any suitable duration and environment for providing a preservable mass quantity of syrup 1239. For example, cold room subsystem 1238 may be any suitable room for holding any suitable number of drums of date sugar concentrate (e.g., date syrup, such as material 1235), such as 500-560 pallets with 4 drums per pallet in 4 layers, which may provide 510-571 tons of syrup.

As mentioned, the pits and any other hard material of pumped combiner material output 205 (e.g., stones, peels, crowns, etc.) may be guided through a different exit of destoner subsystem 206 as second destoner material output 207b (e.g., for receipt by any suitable target, such as any suitable crusher subsystem 240 (e.g., for coarse crushing and/or any other suitable purpose(s))). Crusher subsystem 240 may include any suitable machinery (e.g., a drying plant with any suitable capacity, such as 1,500-2,100 kilograms per hour) for receiving and processing second destoner material output 207b for generating any suitable crusher material output 241 (e.g., coarse crushed date hard material with any suitable moisture (e.g., 11% moisture)). Second filter-press material output 237b (e.g., date fibers, pulp reject, etc.) may be conveyed or otherwise moved by any suitable mover subsystem 238 (e.g., any suitable conveyer belt subsystem (e.g., at 2.1-2.5 tons per hour fiber)) as filter-press material output 239 (e.g., date pulp soft material that may have any suitable moisture (e.g., 85% moisture)).

Any suitable subsystem, such as a dryer subsystem 242, may be provided to receive and further process a combination of crusher material output 241 and filter-press material output 239 (e.g., at a capacity of 2.4-2.5 tons/hour). Dryer subsystem 242 may include any suitable machinery for drying the inputs for providing a first dryer material output 243a (e.g., dried hard date material and dried soft date material) for further processing (e.g., to generate animal feed) and a second dryer material output 243b (e.g., evaporated water (e.g., at 1.5-2.0 tons per hour)).

Any suitable subsystem, such as a grinder subsystem 244, may be provided to receive and further process first dryer material output 243a. Grinder subsystem 242 may include any suitable machinery such as a grinding mill for fine grinding or crushing first dryer material output 243a for providing a grinder material output 245 (e.g., a dry powder with any suitable moisture (e.g., 11% moisture)), which may be bagged by any suitable bagger subsystem 246 (e.g., at 1,250 kilograms per hour) and used for any suitable purpose (e.g., sold as feed for animals or otherwise). As shown, crusher subsystem 240, dryer subsystem 242, grinder subsystem 244, and/or bagger subsystem 246 may be provided by any suitable animal feed plant 247.

The subsystems of system 200 and their associated operations are only illustrative and existing subsystems/operations may be modified or omitted, additional subsystems/operations may be added, and the order of certain subsystems/operations may be altered. Therefore, in some embodiments, a process of creating a date sugar concentrate using system 200 may include pretreatment, extraction of juice, clarification, concentration, and filtration.

As shown in FIG. 3, a subsystem 300 of system 200 (e.g., one, some, or each of the subsystems of system 200 of FIG. 2) may include a processor component 12, a memory component 13, a communications component 14, a sensor 15, an input/output (“I/O”) component 16, a power supply component 17, a structure or housing 11, and/or a bus 18 that may provide one or more wired or wireless communication links or paths for transferring data and/or power to, from, or between various other components of subsystem 300. In some embodiments, one or more components of subsystem 300 may be combined or omitted. Moreover, subsystem 300 may include other components not combined or included in FIG. 3 and/or several instances of the components shown in FIG. 3. For the sake of simplicity, only one of each of the components of subsystem 300 is shown in FIG. 3.

I/O component 16 may include at least one input component (e.g., button, mouse, keyboard, sensor (e.g., sensor 15), lab equipment, etc. (e.g., monitoring module(s)) to receive information or other suitable data from a user/apparatus/material/environment and/or at least one output component (e.g., audio speaker, video display, haptic component, actuator, motor, valve, material source selector component(s), heat/cooling component(s), humidifier component(s), etc. (e.g., controlling module(s))) to provide information or other suitable data to a user/apparatus/material/environment, such as a touch screen that may receive input information through a user's touch of a display screen and that may also provide visual information to a user via that same display screen. Memory 13 may include one or more storage mediums, including for example, a hard-drive, flash memory, magnetic storage, permanent memory such as read-only memory (“ROM”), semi-permanent memory such as random access memory (“RAM”), any other suitable type of storage component, or any combination thereof (e.g., for storing data (e.g., data 19d)). Memory 13 may include suitable logic, circuitry, and/or code that may enable storage of various types of information, such as received data, generated data, code, and/or configuration information.

Communications component 14 may be provided to allow subsystem 300 to communicate with one or more other subsystems 300 using any suitable communications protocol (e.g., via communications network 101). Communications component 14 can be operative to create or connect to any suitable communications network(s). Communications component 14 can provide wireless communications using any suitable short-range or long-range communications protocol, such as Wi-Fi (e.g., an 802.11 protocol), Bluetooth, radio frequency systems (e.g., 1200 MHZ, 2.4 GHz, and 5.6 GHz communication systems), near field communication (“NFC”), Zigbee, wireless local area network (“WLAN”), universal serial bus (“USB”), infrared, protocols used by wireless and cellular telephones and personal e-mail devices, or any other protocol supporting wireless communications. Communications component 14 can also be operative to connect to a wired communications network or directly to another data source wirelessly or via one or more wired connections. Communications component 14 may be a network interface that may include the mechanical, electrical, and/or signaling circuitry for communicating data over physical links that may be coupled to network 101. Such network interface(s) may be configured to transmit and/or receive any suitable data using a variety of different communication protocols, including, but not limited to, TCP/IP, UDP, ATM, synchronous optical networks (“SONET”), any suitable wireless protocols, Frame Relay, Ethernet, Fiber Distributed Data Interface (“FDDI”), and/or the like. In some embodiments, one, some, or each of such network interfaces may be configured to implement one or more virtual network interfaces, such as for Virtual Private Network (“VPN”) access.

Sensor 15 may be any suitable sensor (e.g., monitoring module) that may be configured to sense any suitable data for subsystem 300 (e.g., location-based data via a GPS sensor system, image data, inertia or inertial data, motion data, environmental data, biometric data, apparatus monitoring data, material monitoring data, external environment monitoring data, etc.). Sensor 15 may be a sensor assembly that may include any suitable sensor or any suitable combination of sensors operative to detect any suitable characteristic(s) of subsystem 300 and/or of a user thereof and/or of an associated apparatus and/or its environment/surroundings. Sensor 15 may include any suitable sensor(s), including, but not limited to, one or more of a GPS sensor, wireless communication sensor, image sensor, inertial sensor (e.g., inertial measurement unit (“IMU”)), accelerometer, directional sensor (e.g., compass), gyroscope, motion sensor, pedometer, passive infrared sensor, ultrasonic sensor, microwave sensor, a tomographic motion detector, camera, biometric sensor, light sensor, timer, and/or the like. Sensor 15 may include one or more image sensors for capturing video image data and/or still image data (e.g., sensor 15 may include a rear-facing camera and/or a front-facing camera and/or any other directional camera (e.g., on a gimballed and/or gyrostabilized platform and/or the like) and/or the like). Sensor 15 may include any suitable sensor components or subassemblies for detecting any suitable movement of subsystem 300 and/or of a user thereof and/or of an associated apparatus. For example, sensor 15 may include one or more three-axis acceleration motion sensors (e.g., an accelerometer) that may be operative to detect linear acceleration in three directions (i.e., the x- or left/right direction, the y- or up/down direction, and the z- or forward/backward direction). Any other suitable sensors may also or alternatively be provided by sensor 15 for detecting motion or otherwise at or with subsystem 10, such as any suitable pressure sensors, altimeters, flow sensors, spin sensors, temperature sensors, thermocouples, odor sensors, gas sensors, fluid sensors, humidity sensors, opacity sensors, actuator position sensors, belt rate sensors, electrical resistivity sensors, real density sensors, flow meters, color meters, speed meters, temperature meters, PH meters, refractometers, and/or the like. Using sensor 15, subsystem 300 may be configured to determine a velocity, acceleration, orientation, and/or any other suitable motion attribute of subsystem 300 or an associated apparatus. Sensor 15 may include any suitable sensor components or subassemblies for detecting any suitable biometric data and/or health data and/or the like of a user of user subsystem 300. Sensor 15 may include a microphone, camera, scanner (e.g., a barcode scanner or any other suitable scanner that may obtain product identifying information from a code, such as a linear barcode, a matrix barcode (e.g., a quick response (“QR”) code), or the like), proximity sensor, light detector, temperature sensor, motion sensor, biometric sensor (e.g., a fingerprint reader or other feature (e.g., facial) recognition sensor, which may operate in conjunction with a feature-processing application that may be accessible to subsystem 300 for attempting to authenticate a user), line-in connector for data and/or power, and/or combinations thereof. In some examples, each sensor can be a separate device, while, in other examples, any combination of two or more of the sensors can be included within a single device. While specific examples are provided, it should be appreciated that other sensors can be used and other combinations of sensors can be combined into a single subsystem 300. Sensor 15 may include any suitable sensor components or subassemblies for detecting any suitable characteristics of any suitable condition of the lighting of the environment of subsystem 300 or an associated apparatus or material. In some examples, different sensors can be placed in different locations inside or on the surfaces of subsystem 300 (e.g., some located inside housing 11 and some attached to an attachment mechanism (e.g., a wrist band coupled to a housing of a wearable device), or the like). In other examples, one or more sensors can be worn by a user separately as different parts of a single subsystem 300 or as different devices (e.g., for associating with different respective components of an associated apparatus). In such cases, the sensors can be configured to communicate with subsystem 300 using a wired and/or wireless technology (e.g., via communications component 14). In some examples, sensors can be configured to communicate with each other and/or share data collected from one or more sensors.

Power supply 17 can include any suitable circuitry for receiving and/or generating power, and for providing such power to one or more of the other components of subsystem 300. For example, power supply assembly 17 can be coupled to a power grid (e.g., when subsystem 10 is not acting as a portable device or when a battery of the subsystem is being charged at an electrical outlet with power generated by an electrical power plant). As another example, power supply assembly 17 may be configured to generate power from a natural source (e.g., solar power using solar cells). As another example, power supply assembly 17 can include one or more batteries for providing power (e.g., when subsystem 300 is acting as a portable device). Subsystem 300 may also be provided with a housing 11 that may at least partially enclose one or more of the components of subsystem 300 for protection from debris and other degrading forces external to subsystem 300. Each component of subsystem 300 may be included in the same housing 11 (e.g., as a single unitary device, such as a portable media device or server) and/or different components may be provided in different housings (e.g., a keyboard input component may be provided in a first housing that may be communicatively coupled to a processor component and a display output component that may be provided in a second housing, such as in a desktop computer set-up). In some embodiments, subsystem 300 may include other components not combined or included in those shown or several instances of the components shown.

Processor 12 may be used to run one or more applications, such as an application 19 that may be accessible from memory 13 (e.g., as a portion of data 19d) and/or any other suitable source (e.g., from aby suitable communications network(s) or any other subsystem and an active internet or other suitable data connection). Application 19 may include, but is not limited to, one or more operating system applications, firmware applications, communication applications (e.g., for enabling communication of data between subsystems 300), third party service applications (e.g., sensor applications, social media applications, etc.), internet browsing applications (e.g., for interacting with a website provided by a third party subsystem or other subsystem for enabling subsystem 300 to interact with an online service), application programming interfaces (“APIs”), software development kits (“SDKs”), APS applications (e.g., a web application or a native application for enabling subsystem 300 to interact with an online service), or any other suitable applications. For example, processor 12 may load an application 19 as an interface program to determine how instructions or data received via an input component of I/O component 16 or other component of subsystem 300 (e.g., sensor 15 and/or communications component 14) may manipulate the way in which information may be stored (e.g., in memory 13) and/or provided to the user or associated apparatus via an output component of I/O component 16 and/or to another subsystem via communications component 14. As one example, application 19 may provide a user or subsystem 300 with the ability to interact with a platform of system 200, where application 19 may be a third party application that may be running on subsystem 300 that may be loaded on subsystem 300 (e.g., using communications component 14) via an application market, such as the Apple App Store or Google Play, or that may be accessed via an internet application or web browser (e.g., by Apple Safari or Google Chrome) that may be running on subsystem 300 and that may be pointed to a uniform resource locator (“URL”) whose target or web resource may be managed by or otherwise affiliated with the platform. Processor 12 may include suitable logic, circuitry, and/or code that may enable processing data and/or controlling operations of subsystem 300. In this regard, processor 12 may be enabled to provide control signals to various other components of subsystem 300. Processor 12 may also control transfers of data between various portions of subsystem 300. Processor 12 may further implement an operating system or may otherwise execute code to manage operations of subsystem 300.

Subsystem 300 may be configured to have any physical structure (e.g., by one or more housings 11) that may include, but is not limited to, any suitable portable, mobile, wearable, implantable, rideable, controllable, or hand-held mobile electronic device (e.g., a portable and/or handheld media player), a headset, a helmet, glasses, a wearable, a tablet computer, a laptop computer, a controller, a VR and/or AR and/or MR device, a vehicle, server, sensor system, actuator system, and/or any other machine or device or housing or structure that can be utilized to manage variables of an apparatus. Alternatively, subsystem 300 may not be portable during use, but may instead be generally stationary. Not all of the depicted components may be used in all implementations, however, and one or more implementations may include additional or different components than those shown in FIG. 3. In one or more implementations, one or more of processor 12, memory 13, sensor(s) 15, communications interface or communications component 14, I/O component 16, and/or power supply 17, and/or one or more portions thereof, may be implemented in software (e.g., subroutines and code), may be implemented in hardware (e.g., an application specific integrated circuit (“ASIC”), a field programmable gate array (“FPGA”), a programmable logic device (“PLD”), a controller, a state machine, gated logic, discrete hardware components, or any other suitable devices), and/or a combination of both. Variations in the arrangement and type of the components may be made without departing from the spirit or scope of the claims as set forth herein. Additional components, different components, or fewer components may be provided.

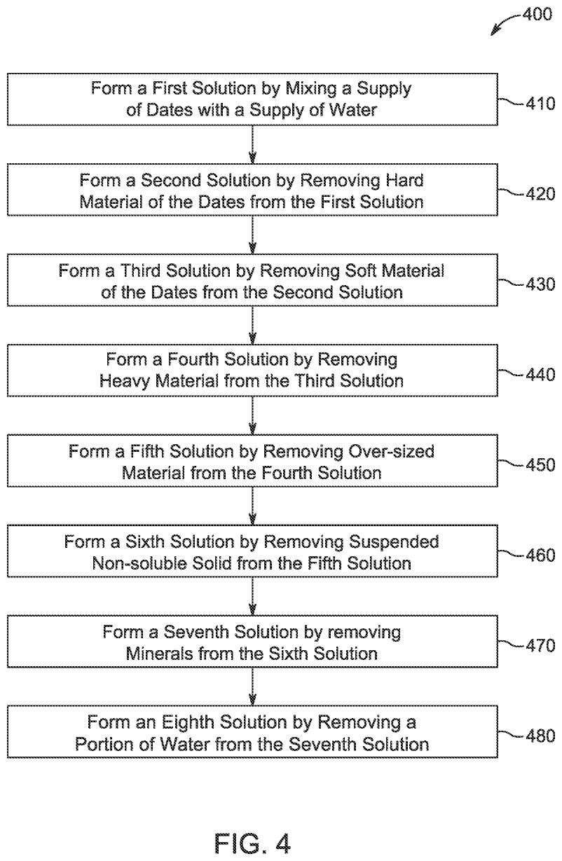

As shown in FIG. 4, an exemplary process 400 may be provided for generating a date product (e.g., date sugar concentrate (e.g., for use in any suitable forms and/or formulations (e.g., gummies, juices, marshmallows, etc.) of the disclosure)), where process 400 may be carried out using any suitable system(s) (e.g., system 200 of FIG. 2). At operation 410 of process 400, a first solution may be formed by mixing a supply of dates with a supply of water (e.g., combiner subsystem 202 may mix dates 201a (e.g., fruit from phoenix dactylifer) with water 202b to form solution 203). At operation 420 of operation 400, a second solution may be formed by removing hard material of the dates from the first solution (e.g., destoner subsystem 208 may remove material 207b from solution 203/205 to form solution 207a (e.g., by removing pits of the dates, seeds of the dates, and/or flesh of the dates using a destoner)). At operation 430 of process 400, a third solution may be formed by removing soft material of the dates from the second solution (e.g., filter-press subsystem 236 may remove material 237b from solution 235 (e.g., an enhanced solution 207a) to form solution 237a (e.g., by removing flesh of the dates, fibers of the dates, and/or calyxes of the dates using a filter-press)). At operation 440 of process 400, a fourth solution may be formed by removing heavy material from the third solution (e.g., separator subsystem 262 may remove material 263b from solution 249/237a to form solution 263a (e.g., by removing heavy particles, sludge, and/or contaminants using a centrifugal separator)). At operation 450 of process 400, a fifth solution may be formed by removing over-sized material from the fourth solution (e.g., micronfiltration subsystem 1240 may remove over-sized material from solution 1240 (e.g., an enhanced solution 263a) to form solution 1241 (e.g., by removing microfibers larger than 1 micron using a pass through filter)). At operation 460 of process 400, a sixth solution may be formed by removing suspended non-soluble solid from the fifth solution (e.g., ultrafiltration subsystem 280 may remove material 281b from solution 279/1241 to form solution 281a (e.g., by removing sub-micron particles, macromolecules, big proteins, vitamins, and/or mineral salts using a network of membrane fitted tubing)). At operation 470 of process 400, a seventh solution may be formed by removing minerals from the sixth solution (e.g., resin filtration subsystem 298 may remove material 299b from solution 295/281a to form solution 299a (e.g., by removing pectins from solution 295/281a, a source of color of solution 295/281, a source of aroma of solution 295/281, and/or a source of taste of solution 295/281 using an adsorbent resin filter)). At operation 480 of process 400, an eighth solution may be formed by removing a portion of water from the seventh solution (e.g., nanofiltration subsystem 1214 may remove material 1215b from solution 1211/299a to form solution 1215a (e.g., by removing at least some pure water using reverse osmosis)). In some embodiments, process 400 may also include forming a ninth solution by evaporating a portion of water from the eighth solution (e.g., using evaporator subsystem 1218) and, in some embodiments, forming a tenth solution by heat sterilizing the ninth solution (e.g., using heat sterilization subsystem 1228). In some embodiments, forming the fifth solution may include removing the over-sized materials from the fourth solution and pasteurizing the result of the removing the over-sized materials from the fourth solution (e.g., using pasteurizing subsystem 259).

The operations shown in process 400 of FIG. 4 are only illustrative and existing operations may be modified or omitted, additional operations may be added, and the order of certain operations may be altered.

The removal of any suspended micro solids (e.g., fiber, ash, undissolved sugars, etc.) by process 100 and/or any process of system 200 and/or from process 400 may result in a very functional and versatile natural sweet liquid with a light syrup consistency. Such a result may be suitable for making naturally sweetened marshmallow, gummies, syrups, sauces, hard candy, toffees, spreads, binder for nuts, seeds, and other food ingredients, chocolate (e.g., when mixed in with cocoa ingredients), baked products (e.g., cookies, biscuits, cakes, breads, pies, etc.), ice cream, and/or the like. Such a filtered product (e.g., resulting from process 100 and/or any process of system 200 and/or from process 400) may be valuable for various reasons, including, but not limited to, (1) the removal of the filtered elements may make the resulting product more predictably reactive when heated (e.g., regular date syrup may become very sticky and impossible to work with and the unfiltered elements may burn well before reaching hard candy temperatures (e.g., regular date syrup may be very dark in color (e.g., black or a reddish dark brown syrup), and may have all the minerals that are present in the raw dates used as well as some of the microfibers, where such regular date syrup may essentially taste like concentrated dates, which may only be desirable in applications where the color is not an issue and the distinct taste of dates is required or otherwise desired)), (2) the flavor of dates is mostly removed along with the filtered elements (e.g., the filtered product is more adaptable to any application outside the scope of dates), and/or (3) many more use cases compared to conventional syrup because of (1) and (2). Therefore, the result or filtered product of process 100 and and/or any process of system 200 and/or process 400 may be referred to herein as a decolorized date syrup, date liquid sugar, date juice concentrate, and/or date sugar concentrate.

These processes are configured to remove a big portion of the color and “datey” flavor, which may result in a golden-translucent liquid with a syrupy consistency. This resultant date sugar concentrate may be usable as a natural and neutral sweetener in virtually any application (e.g., baking, confectionary, beverage, etc.). Other fruit varieties (e.g., other than dates), even if run through the same process(es) for color clarification, may still largely carry a flavor influence of the fruit that was processed (e.g., white grape syrup, although it is a clear, relatively colorless syrup, still strongly tastes of grapes). The composition of the decolorized date syrup, date liquid sugar, date juice concentrate, and/or date sugar concentrate of this disclosure may include dates and water (e.g., these may be the only two ingredients directly used and processed to generate the result of process 100 and/or any process of system 200 and/or process 400). The resulting concentrate may include sugar extracted from dates (e.g., about 68% or 70% by volume) and may be suspended in water (e.g., about 30% by volume). In some embodiments, the date sugar includes only glucose and fructose as naturally found in the date fruit. The ratio of glucose to fructose may be about 50/50. Sometimes, depending on the maturity of the raw dates, some trace amounts of sucrose may be present. Once, the ash and mineral content of dates are able to be efficiently removed from the solution (e.g., ash and mineral content may contain the color pigments and taste of the date), the resulting syrup/concentrate may be used in an array of new applications, such as date gummies and marshmallows.

Gummies may include a liquid (e.g., date sugar concentrate), sensory ingredients for color and flavor as may be desired, a hydrophilic long-chain polymer (e.g., to solidify the mixture into form), functional ingredients (e.g., one or more acidic compounds to facilitate the functionality of pectin (e.g., if used)), and/or a fruit powder (e.g., to boost flavor and/or provide for a stronger form). Marshmallows may be predominantly date sugar concentrate and gelatin, which may be heated and aerated, and then formed. Once the ash and mineral content naturally found in dates is removed from this concentrate, the resulting natural sugar solution may be heated to higher temperatures (e.g., wherein the ash and mineral content would normally burn and release a burnt taste making the product unmarketable, and wherein the ash and mineral content would also cause the product, once solidified, to become very tacky and sticky, which is highly undesirable). Moreover, the removal of flavor allows any other generic fruit flavor to be added without it clashing with date flavor, which is often regarded as distinct and overbearing.

The combination(s) and sequence(s) of operations of process 100 and/or any process of system 200 and/or process 400 are unique and novel for concentrating date sugar. Conventionally, the idea behind fruit concentrate is to capture and intensify the essence (e.g., flavor and aroma) of the fruit. The date sugar concentrate of this disclosure may be designed largely to remove the flavor and aroma of dates (e.g., in order to leave only the natural sweetness). By doing so, this becomes a much more adaptable and versatile product that is usable in a wide array of applications. Additionally, by removing the ash and mineral content, the product becomes much more resilient to heat. Instead of the ash and minerals burning and releasing a burnt taste and odor at relatively modest temperatures (e.g., 100-110 degrees Celsius), higher temperatures are able to be used with this product such that it may be used in candy and confectionary applications (e.g., applications that may require heating sugars to about 145 degrees Celsius). Moreover, the resulting neutrality of sweetness allows this product to be used as a sweetener in other beverages, such as mango juice, apple juice, berry juice, and many more, and/or to be used in baking applications where a neutral sweetness is desired. The unique combination of method, machinery, and raw input (e.g., dates) might typically be regarded as counterproductive, as the flavor of the dates is largely eliminated. However, this methodology has been adopted and developed entirely to achieve such end results (e.g., a product that may be heated to high temperatures and/or a product with a neutrality of sweetness).

As just one example, a marshmallow product may be manufactured to include any suitable ingredients, such as a first ingredient of decolorized date syrup to function as a base, natural sweetener that may provide ˜89.3% of the product, a second ingredient of gelatin (e.g., ˜50% by volume) to function as a hydrophilic long chain polymer that may provide ˜10% of the product, a third ingredient of an oil or alcohol based flavor of natural or nature identical source (e.g., mainly vanilla) to function as a flavoring agent that may provide ˜0.20 of the product, a fourth ingredient of a liquid food grade dye to function as an optional coloring agent that may provide ˜0.20 of the product, and a fifth ingredient of a cocoa powder to function as an optional cocoa powder coating (e.g., sanding) to coat the exterior of the marshmallow and provide additional flavor that may provide ˜0.30 of the product. Any suitable process may be utilized to manufacture such a product with any suitable operations, including but not limited to, a first operation for heating the syrup (e.g., to 230-240 degrees F.), a second operation for transferring the result to a mixer where the hydrated gelatin may be added and mixed in, a third operation for using an aerator to beat air into the mixture, a fourth operation for extruding that may eject the mixture (e.g., with the air) hot in a chosen shape, a fifth operation for cooling and cutting the marshmallow forms for remain in a chosen shape, and/or a sixth operation for optionally coating/sanding in cocoa powder as an alternative to starch sanding, which may ensure that the forms do not stick together. These operations are only illustrative and existing operations may be modified or omitted, additional operations may be added, and the order of certain operations may be altered.

As just one other example, a gelatin-based gummy product may be manufactured to include any suitable ingredients, such as a first ingredient of decolorized date syrup to function as a base, natural sweetener that may provide ˜79.6% of the product, a second ingredient of apple juice concentrate (e.g., 2.5% malic acid) to function as a hydrating ingredient (e.g., to assist in dispersion of solid ingredients) that may provide ˜10% of the product, a third ingredient of gelatin (e.g., 50% by volume, hydrated) to function as a hydrophilic long chain polymer that may provide ˜10% of the product, a fourth ingredient of oil or alcohol based flavor of natural or nature identical source (e.g., mainly vanilla) to function as a flavoring agent that may provide ˜0.2% of the product, and a fifth ingredient of powder or liquid form from natural or processed source to function as a coloring agent that may provide ˜0.2% of the product (e.g., possibly using freeze dried fruit powders as a natural source for both color and flavor). Any suitable process may be utilized to manufacture such a product with any suitable operations, including but not limited to, a first operation for heating the syrup (e.g., to 230-240 degrees F.), a second operation for separately mixing the hydrated gelatin with the apple juice concentrate, a third operation for combining both mixtures and continuing high sheer mixing until the gelatin is fully dissolved/disbursed, a fourth operation for adding flavoring and coloring agents while continuing mixing, a fifth operation for transferring the result to molds, a sixth operation for refrigerating the result for a few hours before de-molding, and/or a seventh operation for polishing the in rotary drum with care. These operations are only illustrative and existing operations may be modified or omitted, additional operations may be added, and the order of certain operations may be altered.

As just one other example, a pectin-based (e.g., vegan) gummy product may be manufactured to include any suitable ingredients, such as a first ingredient of decolorized date syrup to function as a base, natural sweetener that may provide ˜81.85% of the product, a second ingredient of apple juice concentrate (e.g., 2.5% malic acid) to function as a hydrating ingredient (e.g., to assist in dispersion of solid ingredients) that may provide ˜10% of the product, a third ingredient of citric acid (e.g., 50% solution) to function as an acidity regulator that may optimize the performance of the pectin to added that may provide ˜2% of the product, a fourth ingredient of sodium citrate to function as an acidity regulator that may provide ˜0.75% of the product, a fifth ingredient of pectin JD 470 to function as a hydrophilic long chain polymer that may provide ˜5% of the product, a sixth ingredient of oil or alcohol based flavor of natural or nature identical source (e.g., mainly vanilla) to function as a flavoring agent that may provide ˜0.2% of the product, and a seventh ingredient of powder or liquid form from natural or processed source to function as a coloring agent that may provide ˜0.2% of the product (e.g., possibly using freeze dried fruit powders as a natural source for both color and flavor). Any suitable process may be utilized to manufacture such a product with any suitable operations, including but not limited to, a first operation for dissolving pectin into date syrup, a second operation for mixing the remaining ingredients and heating them (e.g., to 175 degrees F.), a third operation for combining both mixtures and continuing mixing until completely homogenous and temperature reaches the desired heat (e.g., 175 degrees F.), a fourth operation for molding and refrigerating to set, a fifth operation for de-molding then coating with wax/oil, a sixth operation for refrigerating the result until cured, and/or a seventh operation for packaging the result. These operations are only illustrative and existing operations may be modified or omitted, additional operations may be added, and the order of certain operations may be altered.

Therefore, any suitable gummy date products may be created, such as gummy date-bears, which may be made almost entirely from dates, with colors and/or flavors from natural sources, any suitable net weight (e.g., 1.42 oz (40 g)), any suitable nutritional information/per 50 g, such as energy=0%, total fat=0% of which 0% is saturated, cholesterol=0%, potassium=0%, sodium=0%, total carbohydrates=0%, dietary fibers=0%, sugars=0%, protein=0%, vitamin c=0%, riboflavin=0%, vitamin b6=0%, phosphorus=0%, calcium=0%, niacin=0%, pantothenic ac=0%, magnesium=0%, such as based on a daily 2,000 calorie diet, where ingredients may include dates, freeze dried fruit powder, gelatin (gelling agent), and carnauba wax (glazing agent). The product may be made in the United Arab Emirates (“UAE”) and may be stored in a cool dry place and away from sunlight. Therefore, the product may be a sweet treat made using all-natural sweetening, flavoring, and coloring ingredients. Thus, dates may be used as a cornerstone ingredient replacing processed foods for providing a truly guilt free product.

As just one other example, a date pancake (e.g., maple) syrup product may be manufactured to include any suitable ingredients, such as a first ingredient of decolorized date syrup to function as a base, natural sweetener that may provide ˜99.58% of the product, a second ingredient of maple flavor to function as a flavor source of natural or nature identical source that may provide ˜0.2% of the product, a third ingredient of guar gum to function as a thickening agent that may provide ˜0.2% of the product, and a fourth ingredient of preservative (e.g., potassium sorbate or netamax) to function as a preservative that may provide ˜0.02% of the product. Any suitable process may be utilized to manufacture such a product with any suitable operations, including but not limited to, a first operation for heating of the date syrup (e.g., to 175 degrees F.), a second operation for adding the previously dissolved ingredients in 5 parts date syrup, a third operation for continued heating until the mixture is homogenous and the heat is equally distributed, and/or a fourth operation for filling the result in bottles. These operations are only illustrative and existing operations may be modified or omitted, additional operations may be added, and the order of certain operations may be altered.

Therefore, any suitable date syrup products may be created, such as date-maple syrup, which may be made almost entirely from dates, with zero added sugar, only from natural sources, naturally flavored, no sugar alcohols, no artificial sweeteners, any suitable net weight (e.g., 11 oz (310 g)), a nutritional information/serving size=1 tblsp. (40 g) amount per serving=energy 126 kcal % daily value based on 2,000 calorie diet of protein=260 mg=0%, total fat=0 g=0%, of which saturated=0 g=0%, cholesterol=0 mg=0%, total carbohydrates=31 g=13%, dietary fibers=1 g=3%, sugars=30 g=12%, sodium=6 mg=1%, potassium=2 mg=0%, calcium=2 mg=0%, vitamin c=0 mg=0%, vitamin b6=0 mg=0%, phosphorus=0 mg=0%, magnesium=1 mg=0%, vitamin b2 (riboflavin)=0 mg=0%, vitamin b3 (niacin)=0 mg=0%, vitamin b5 (pantothenic acid)=0 mg=0%, where ingredients may include dates (more than 90%), nature identical flavor, guar gum (thickener), potassium sorbate (preservative). made in the UAE and may be stored in a cool dry place and away from sunlight.

As just one other example, a date chocolate syrup product may be manufactured to include any suitable ingredients, such as a first ingredient of decolorized date syrup to function as a base, natural sweetener that may provide ˜94.78% of the product, a second ingredient of cocoa powder to function as a coloring and flavoring agent of natural source that may provide ˜4.0% of the product, a third ingredient of flavoring agent to function as a flavoring agent of natural or nature identical source that may provide ˜0.15% of the product, a fourth ingredient of masking flavor to function as a masking flavor to reduce sweetness that may provide ˜0.40% of the product, a fifth ingredient of salt to function as a flavor component to assist the cocoa flavor potency that may provide ˜0.45% of the product, a sixth ingredient of guar gum to function as a thickening agent that may provide ˜0.20% of the product, and a seventh ingredient of preservative (potassium sorbate or netamax) to function as a preservative that may provide ˜0.02% of the product. Any suitable process may be utilized to manufacture such a product with any suitable operations, including but not limited to, a first operation for heating a majority (e.g., 80%) of the date syrup required (e.g., to 175 degrees F.), a second operation for adding the cocoa powder and dissolving, a third operation for mixing remaining ingredients with the remaining (e.g., 20%) of the date syrup, a fourth operation for combining both mixtures and mixing until homogenous and temperature reaches a suitable temperature (e.g., 175 degrees F.) and held for any suitable amount of time (e.g., 10 minutes), and/or a fifth operation for filling the result in bottles. These operations are only illustrative and existing operations may be modified or omitted, additional operations may be added, and the order of certain operations may be altered.

Therefore, any suitable date syrup products may be created, such as date-chocolate syrup, which may be made almost entirely from dates, with zero added sugar, only from natural sources, naturally flavored, no sugar alcohols, no artificial sweeteners, any suitable net weight (e.g., 11 oz (310 g)), with nutritional information/serving size=1 tblsp. (40 g), amount per serving=energy 125 kcal, % daily value based on a daily 2,000 calorie diet of protein=700 mg=1%, total fat=0 g=0%, of which saturated=0 g=0%, cholesterol=0 mg=0%, total carbohydrates=30 g=12%, dietary fibers=1 g=4%, sugars=29 g=11%, sodium=94 mg=4%, potassium=49 mg=1%, calcium=7 mg=1%, vitamin c=1 mg=1%, vitamin b6=0 mg=0%, phosphorus=13 mg=1%, magnesium=9 mg=2%, vitamin b2 (riboflavin)=0 mg=0%, vitamin b3 (niacin)=0 mg=0%, vitamin b5 (pantothenic acid)=0 mg=0%, with any suitable ingredients such as dates (more than 90%), cocoa powder, nature identical flavor, salt, guar gum (thickener), and potassium sorbate (preservative), which may be. made in the UAE and may be stored in a cool dry place and away from sunlight.

In some embodiments, the result or filtered product of process 100 and and/or any process of system 200 (e.g., material 1235) and/or of process 400 may be referred to herein as a decolorized date syrup, date liquid sugar, date juice concentrate, and/or date sugar concentrate, and may be used in any suitable applications. The product may have any suitable composition, such as 15-91% water (e.g., Chemical Abstracts Service Registry Number (“CASRN”) 7732-18-5), 2-42% fructose (e.g., CASRN 57-48-7), 2-44% glucose (e.g., CASRN 50-99-7), 0-4% malic acid (e.g., CASRN 97-67-6), 0-12% citric acid (e.g., CASRN 77-92-9), and 0-3% ash, which may provide any suitable appearance, such as a gold to reddish brown viscous liquid or a light golden yellow to golden brown viscous liquid (e.g., ICUMSA Gs9/1/2/3-8 (2011); <1,100 IU), and/or which may provide any suitable odor (if any), such as sweet and/or mild lactonic and/or non-fruity, and may be soluble in water. The final product may vary depending on the variety, seasonal factors, and condition of the raw dates available/used (e.g., at combiner material input 201a). However, in some embodiments, the color of the product may not be over 1,100 IU (e.g., an acceptable limit that may be set by system 200). In some embodiments, the product may have any suitable chemical properties, such as 72-75 Brix (Degrees BX), 3.80-4.80 pH, 0.03-0.10 acidity (%), 27.00-38.00 or 30.00-36.00 glucose (%), 30.00-36.00 fructose (%), 1.00-3.00 protein (%), and/or trace amounts (if any (e.g., one part per million)) sucrose (%) (e.g., as may be dependent on presence in raw dates and seasonal factors). In some embodiments, it may have any suitable microbiological properties, such as <100 yeast (cfu/g), <100 mold (cfu/g), no E. Coli (cfu/g), no salmonella (cfu/g), and no Listeria monocytogenes (cfu/g). No contaminants may be present in the product, except for, in some embodiments, 3 ppb of Ochratoxin A. Storage conditions may influence the color maturity of the product. It may be recommended to always keep the product away from direct sunlight and in its original packaging until use. Color evolution of the product may be influenced by product shelf life in relation to storage temperatures and conditions. The product color may gradually mature and darken with time if stored under ambient conditions. For example, when stored at 2-8° Celsius, the color evoluation may be about 12 months from production, and/or, when stored at 18-22° Celsius, the color evoluation may be about 4 months from production. Heavy metal residues do not exceed the limits of C.E. Reg. 629/2008. The product is free of allergens, according to C.E. Reg. 1169/2011. The product is Dioxin-free and does not come in contact with contaminated sources of Dioxin. The product Free of GMO according to C.E. Reg. 1829/2003 and 1830/2003, it does not derive and does not contain GMO ingredients and it is not processed by using GMO products.

Although “gummy” may be used to describe one or more certain forms and/or formulations herein, other suitable forms and/or formulations may be utilized and/or provided according to the concepts of the disclosure (e.g., manufactured using the date sugar concentrate product of the disclosure), including, but not limited to, juices, marshmallows, syrups, sauces, hard candy, toffees, spreads, binder for nuts, seeds, and other food ingredients, chocolate (e.g., when mixed in with cocoa ingredients), baked products (e.g., cookies, biscuits, cakes, breads, pies, etc.), ice cream, nutritional and/or wellness vehicles, and/or the like.

One, some, or all of the processes described with respect to FIGS. 1-4 and otherwise may each be at least partially implemented by software, but may also be implemented in hardware, firmware, or any combination of software, hardware, and firmware. Instructions for performing these processes may also be embodied as machine- or computer-readable code recorded on a machine- or computer-readable medium. In some embodiments, the computer-readable medium may be a non-transitory computer-readable medium. Examples of such a non-transitory computer-readable medium include but are not limited to a read-only memory, a random-access memory, a flash memory, a CD-ROM, a DVD, a magnetic tape, a removable memory card, and a data storage device. In other embodiments, the computer-readable medium may be a transitory computer-readable medium. In such embodiments, the transitory computer-readable medium can be distributed over network-coupled computer systems so that the computer-readable code is stored and executed in a distributed fashion. For example, such a transitory computer-readable medium may be communicated from one subsystem to another directly or via any suitable network or bus or the like. Such a transitory computer-readable medium may embody computer-readable code, instructions, data structures, program modules, or other data in a modulated data signal, such as a carrier wave or other transport mechanism, and may include any information delivery media. A modulated data signal may be a signal that has one or more of its characteristics set or changed in such a manner as to encode information in the signal.

Any, each, or at least one module or component or subsystem of the disclosure may be provided as a software construct, firmware construct, one or more hardware components, or a combination thereof. For example, any, each, or at least one module or component or subsystem of any suitable system may be described in the general context of computer-executable instructions, such as program modules, that may be executed by one or more computers or other devices. Generally, a program module may include one or more routines, programs, objects, components, and/or data structures that may perform one or more particular tasks or that may implement one or more particular abstract data types. The number, configuration, functionality, and interconnection of the modules and components and subsystems of such a system are only illustrative, and that the number, configuration, functionality, and interconnection of existing modules, components, and/or subsystems may be modified or omitted, additional modules, components, and/or subsystems may be added, and the interconnection of certain modules, components, and/or subsystems may be altered.

As may be used in this specification and any claims of this application, the terms “base station,” “receiver,” “computer,” “server,” “processor,” and “memory” may all refer to electronic or other technological devices. These terms exclude people or groups of people. For the purposes of the specification, the terms “display” or “displaying” means displaying on an electronic device.

As may be used herein, the terms “computer,” “personal computer,” “device,” “computing device,” “router device,” and “controller device” may refer to any programmable computer system that is known or that will be developed in the future. In certain embodiments, a computer will be coupled to a network, such as described herein. A computer system may be configured with processor-executable software instructions to perform the processes described herein. Such computing devices may be mobile devices, such as a mobile telephone, data assistant, tablet computer, or other such mobile device. Alternatively, such computing devices may not be mobile (e.g., in at least certain use cases), such as in the case of server computers, desktop computing systems, or systems integrated with non-mobile components.

As may be used herein, the terms “component,” “module,” and “system,” are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server may be a component. One or more components may reside within a process and/or thread of execution and a component may be localized on one computer and/or distributed between two or more computers.

The predicate words “configured to,” “operable to,” “operative to,” and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation or the processor being operative to monitor and control the operation. Likewise, a processor configured to execute code can be construed as a processor programmed to execute code or operable to execute code or operative to execute code.

As used herein, the term “based on” may be used to describe one or more factors that may affect a determination. However, this term does not exclude the possibility that additional factors may affect the determination. For example, a determination may be solely based on specified factors or based on the specified factors as well as other, unspecified factors. The phrase “determine A based on B” specifies that B is a factor that is used to determine A or that affects the determination of A. However, this phrase does not exclude that the determination of A may also be based on some other factor, such as C. This phrase is also intended to cover an embodiment in which A may be determined based solely on B. As used herein, the phrase “based on” may be synonymous with the phrase “based at least in part on.”

As used herein, the phrase “in response to” may be used to describe one or more factors that trigger an effect. This phrase does not exclude the possibility that additional factors may affect or otherwise trigger the effect. For example, an effect may be solely in response to those factors, or may be in response to the specified factors as well as other, unspecified factors. The phrase “perform A in response to B” specifies that B is a factor that triggers the performance of A. However, this phrase does not foreclose that performing A may also be in response to some other factor, such as C. This phrase is also intended to cover an embodiment in which A is performed solely in response to B.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. The term “and/or” as used herein refers to and encompasses any and all possible combinations of one or more of the associated listed items. As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” may each refer to only A, only B, or only C; any combination of A, B, and C; and/or at least one of each of A, B, and C. The terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. When used in the claims, the term “or” is used as an inclusive or and not as an exclusive or. For example, the phrase “at least one of x, y, or z” means any one of x, y, and z, as well as any combination thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter/neutral gender (e.g., her and its and they) and vice versa. Headings and subheadings, if any, are used for convenience only and do not limit the subject disclosure.

While there have been described date sugar concentrates and methods for using and making the same, many changes may be made therein without departing from the spirit and scope of the subject matter described herein in any way. Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. It is also to be understood that various directional and orientational terms, such as “left” and “right,” “up” and “down,” “front” and “back” and “rear,” “top” and “bottom” and “side,” “above” and “below,” “length” and “width” and “thickness” and “diameter” and “cross-section” and “longitudinal,” “X-” and “Y-” and “Z-,” and/or the like, may be used herein only for convenience, and that no fixed or absolute directional or orientational limitations are intended by the use of these terms. For example, the components of an apparatus can have any desired orientation. If reoriented, different directional or orientational terms may need to be used in their description, but that will not alter their fundamental nature as within the scope and spirit of the disclosure.

Therefore, those skilled in the art will appreciate that the concepts of the disclosure can be practiced by other than the described embodiments, which are presented for purposes of illustration rather than of limitation.

1. A method of generating a date product comprising:
forming a first solution by mixing a supply of dates with a supply of water;
forming a second solution by removing hard material of the dates from the first solution;
forming a third solution by removing soft material of the dates from the second solution;
forming a fourth solution by removing heavy material from the third solution;
forming a fifth solution by removing over-sized material from the fourth solution;
forming a sixth solution by removing suspended non-soluble solid from the fifth solution;
forming a seventh solution by removing minerals from the sixth solution; and
forming an eighth solution by removing a portion of water from the seventh solution.

2. The method of claim 1, wherein the supply of dates comprises fruit from Phoenix dactylifera.

3. The method of claim 1, wherein the removing hard material of the dates from the first solution comprises removing at least one of the following using a destoner:
pits of the dates;
seeds of the dates; or
flesh of the dates.

4. The method of claim 1, wherein the removing soft material of the dates from the second solution comprises removing at least one of the following using a filter-press:
flesh of the dates;
fibers of the dates; or
calyxes of the dates.

5. The method of claim 1, wherein the removing heavy material from the third solution comprises removing at least one of the following using a centrifugal separator:
heavy particles;
sludge; or
contaminants.

6. The method of claim 1, wherein the removing over-sized material from the fourth solution comprises removing microfibers larger than 1 micron using a pass through filter.

7. The method of claim 1, wherein the removing suspended non-soluble solid from the fifth solution comprises removing at least one of the following using a network of membrane fitted tubing:
sub-micron particles;
macromolecules;
big proteins;
vitamins; or
mineral salts.

8. The method of claim 1, wherein the removing minerals from the sixth solution comprises removing at least one of the following using an adsorbent resin filter:
pectins of the sixth solution;
a source of color of the sixth solution;
a source of aroma of the sixth solution; or
a source of taste of the sixth solution.

9. The method of claim 1, wherein the removing a portion of water from the seventh solution comprises removing at least some pure water using reverse osmosis.

10. The method of claim 1, further comprising forming a ninth solution by evaporating a portion of water from the eighth solution.

11. The method of claim 10, further comprising forming a tenth solution by heat sterilizing the ninth solution.

12. The method of claim 1, wherein the forming the fifth solution comprises:
removing the over-sized materials from the fourth solution; and
pasteurizing the result of the removing the over-sized materials from the fourth solution.

13. A date syrup comprising:
water; and
date sugar of at least 70° Brix in the water.

14. The date syrup of claim 13, wherein the date syrup has a turbidity of less than 15 nephelometric turbidity units.

15. A date sugar concentrate comprising:
date sugar in an amount of at least 68% by volume; and
water in an amount of at least 30% by volume.

16. The date sugar concentrate of claim 15, wherein the date sugar concentrate has a turbidity of less than 15 nephelometric turbidity units.

17. The date sugar concentrate of claim 15, wherein the date sugar concentrate comprises neither ash nor mineral content of dates.

18. A semi-solid form for oral administration, comprising:
the date sugar concentrate of claim 15 in an amount of at least 60% by volume; and
a hydrophilic long-chain polymer.

19. The semi-solid form of claim 18, wherein:
the semi-solid form is a marshmallow; and
the semi-solid form comprises:
the date sugar concentrate in an amount of at least 80% by volume; and
the hydrophilic long-chain polymer in an amount of at least 10% by volume.

20. The semi-solid form of claim 18, wherein:
the semi-solid form is a gummy; and
the semi-solid form comprises:
the date sugar concentrate in an amount of at least 75% by volume; and
the hydrophilic long-chain polymer in an amount of at least 5% by volume.

21-50. (canceled)
